(19) United States
(12) Patent Application Publication
(10) Pub. No.: US 2015/0366798 A1
(43) Pub. Date: Dec. 24, 2015

(54) PHARMACEUTICAL ADHESIVE COMPOSITIONS FOR TREATMENT OF EPISTAXIS AND METHODS OF USE THEREOF

(71) Applicants: Evgenia Lozinsky, Beer-Sheva (IL); Eran Eilat, Herzliya (IL)

(72) Inventors: Evgenia Lozinsky, Beer-Sheva (IL); Eran Eilat, Herzliya (IL)

(21) Appl. No.: 14/702,376

(22) Filed: May 1, 2015

Related U.S. Application Data
(60) Provisional application No. 62/014,572, filed on Jun. 19, 2014.

(51) Int. Cl. A61K 9/00; A61K 31/196; A61K 47/24; A61K 47/34; A61L 26/00; A61K 47/10; A61K 47/32; A61K 47/38; A61K 47/20; A61K 31/4174; A61K 31/137; A61K 47/12

(52) U.S. Cl. CPC A61K 9/0043; A61K 31/137; A61K 31/196; A61K 47/24; A61K 47/34; A61K 47/12; A61K 47/10; A61K 47/32; A61K 47/38; A61K 47/20; A61K 31/4174; A61L 26/009; A61L 26/0076; A61L 26/0066; A61L 26/0052; A61L 26/0023; A61L 26/0019; A61L 26/0014

(57) ABSTRACT

The present invention provides liquid adhesive compositions including at least one silicone resin film forming agent, at least one surfactant, at least one volatile solvent, water and at least one vasoconstrictor, where the compositions are in the form of an emulsion. The invention further provides methods for treating any type of nose bleeding, including Epistaxis, including administering the liquid adhesive compositions to the nose, in the affected nostril. Further provided is a kit, including the liquid adhesive composition and a container-applicator device.

### RELATED APPLICATION

This application claims the benefit of and priority to U.S. Provisional Application No. 62/014,572, filed Jun. 19, 2014, which is incorporated herein by reference in its entirety.

### BACKGROUND OF THE INVENTION

Epistaxis or nosebleed, the term used to describe hemorrhage from the nose, is relatively common, usually noticed when the blood drains out through the nostrils. There are two types: anterior (the most common), and posterior (less common, more likely to require medical attention). Sometimes in more severe cases, the blood can come up the nasolacrimal duct and out from the eye. Fresh blood and clotted blood can also flow down into the stomach and cause nausea and vomiting.

Nosebleeds are due to the rupture of a blood vessel within the richly perfused nasal mucosa. Rupture may be spontaneous or initiated by trauma. Nosebleeds are reported in up to 60% of the population with peak incidences in those under the age of ten and over the age of 50 and appear to occur in males more than females. An increase in blood pressure (e.g. due to general hypertension) tends to increase the duration of spontaneous epistaxis. Anticoagulant medication and disorders of blood clotting can promote and prolong bleeding. Spontaneous epistaxis is more common in the elderly as the nasal mucosa (lining) becomes dry and thin and blood pressure tends to be higher. The elderly are also more prone to prolonged nose bleeds as their blood vessels are less able to constrict and control the bleeding.

The vast majority of nose bleeds occur in the anterior (front) part of the nose from the nasal septum. This area is richly endowed with blood vessels (Kiesselbach's plexus). This region is also known as Little's area. Bleeding farther back in the nose is known as a posterior bleed and is usually due to bleeding from Woodruff s plexus, a venous plexus situated in the posterior part of inferior meatus. Posterior bleeds are often prolonged and difficult to control. They can be associated with bleeding from both nostrils and with a greater flow of blood into the mouth.

### SUMMARY OF THE INVENTION

The present invention provides adhesive compositions comprising vasoconstrictors and uses thereof for treating Epistaxis or nosebleed, mostly caused by injury to the nose, hemophilia, upper respiratory infection, hypertension, antiplatelet medication, foreign body, insufflated drugs, barotrauma, nasal surgery, nasal sprays, allergic reactions or combinations thereof. The liquid adhesive compositions of the present invention are also useful for treating other types of Epistaxis.

Liquid adhesive compositions comprise polar solvents which enable the incorporation of the medicaments into the liquid adhesive.

The major disadvantage of such liquid adhesive compositions comprising polar solvents, e.g. ethanol, is their stinging effect when applied to the mucosal surface.

It was now surprisingly discovered that including an aqueous phase in the liquid adhesive compositions of the present invention enables the dissolution and substantially homogeneous distribution of the vasoconstrictors in the composition. Inclusion of water in the liquid adhesive composition obviates or reduces the use of polar solvents, prevents or improves the stinging effect and hence improves the compliance of the subject to be treated. The novel compositions of the instant invention comprise in addition to water also silicon acrylate and silicon surfactants, which enable the formation of stable emulsions. It was further discovered that the liquid adhesive compositions of the present invention form a film on the bleeding nasal surfaces and thus provide a protective coating on irritated nasal tissue. In addition, the liquid adhesive compositions of the present invention provide sustained release of the vasoconstrictor from the liquid adhesive, thus leading to enhanced healing of the affected areas. According to a first aspect, the present invention provides a pharmaceutical liquid adhesive composition comprising: (i) a silicone resin continuous film forming agent selected from the group of silicone acrylates, silicon surfactant(s), and combinations thereof; (ii) at least one surfactant, (iii) a volatile solvent; (iv) water; and (v) at least one vasoconstrictor, wherein the composition is in the form of an emulsion. According to another embodiment, the emulsion is a water-in-oil emulsion.

According to another aspect, the volatile solvent is a volatile silicone solvent.

According to one embodiment, the silicon resin continuous film is silicon acrylate.

According to another embodiment, the silicone resin continuous film is a siloxysilicate like trimethylsiloxysilicate.

In some embodiments, the composition of the present invention includes 10.0% to 40.0% of at least one silicon resin film forming agent. In some embodiments, the composition of the present invention includes 15.0% to 40.0% of at least one silicon resin film forming agent. In some embodiments, the composition of the present invention includes 20.0% to 40.0% of at least one silicon resin film forming agent. In some embodiments, the composition of the present invention includes 25.0% to 40.0% of at least one silicon resin film forming agent. In some embodiments, the composition of the present invention includes 30.0% to 40.0% of at least one silicon resin film forming agent. In some embodiments, the composition of the present invention includes 35.0% to 40.0% of at least one silicon resin film forming agent. In some embodiments, the composition of the present invention includes 10.0% to 35.0% of at least one silicon resin film forming agent. In some embodiments, the composition of the present invention includes 10.0% to 30.0% of at least one silicon resin film forming agent. In some embodiments, the composition of the present invention includes 10.0% to 25.0% of at least one silicon resin film forming agent. In some embodiments, the composition of the present invention includes 10.0% to 20.0% of at least one silicon resin film forming agent. In some embodiments, the composition of the present invention includes 10.0% to 15.0% of at least one silicon resin film forming agent.

In some embodiments, the at least one silicon resin film forming agent is selected from the group consisting of siloxysilicates, silicone acrylates and combinations thereof.

According to some embodiments, the at least one surfactant is silicone surfactant. In some embodiments, the composition of the present invention includes from between 30% to 80% of a silicone surfactant. In some embodiments, the composition of the present invention includes from between 35% to 80% of a silicone surfactant. In some embodiments, the composition of the present invention includes from between 40% to 80% of a silicone surfactant. In some embodiments, the composition of the present invention includes from between 45% to 80% of a silicone surfactant. In some embodiments, the composition of the present invention includes from between 50% to 80% of a silicone surfactant. In some embodiments, the composition of the present invention includes from between 55% to 80% of a silicone surfactant. In some embodiments, the composition of the present invention includes from between 60% to 80% of a silicone surfactant. In some embodiments, the composition of the present invention includes from between 65% to 80% of a silicone surfactant. In some embodiments, the composition of the present invention includes from between 70% to 80% of a silicone surfactant. In some embodiments, the composition of the present invention includes from between 75% to 80% of a silicone surfactant. In some embodiments, the composition of the present invention includes from between 30% to 75% of a silicone surfactant. In some embodiments, the composition of the present invention includes from between 30% to 70% of a silicone surfactant. In some embodiments, the composition of the present invention includes from between 30% to 65% of a silicone surfactant. In some embodiments, the composition of the present invention includes from between 30% to 60% of a silicone surfactant. In some embodiments, the composition of the present invention includes from between 30% to 55% of a silicone surfactant. In some embodiments, the composition of the present invention includes from between 30% to 50% of a silicone surfactant. In some embodiments, the composition of the present invention includes from between 30% to 45% of a silicone surfactant. In some embodiments, the composition of the present invention includes from between 30% to 40% of a silicone surfactant. In some embodiments, the composition of the present invention includes from between 30% to 35% of a silicone surfactant. In some embodiments, the composition of the present invention includes from between 35% to 75% of a silicone surfactant. In some embodiments, the composition of the present invention includes from between 40% to 70% of a silicone surfactant. In some embodiments, the composition of the present invention includes from between 45% to 65% of a silicone surfactant. In some embodiments, the composition of the present invention includes from between 50% to 60% of a silicone surfactant.

According to some embodiments, the organosilicon surfactant comprises alkyl- and alkoxy-dimethicone copolyol. According to further embodiments, the alkyl- and alkoxy-dimethicone copolyol is cetyl dimethicone copolyol. According to a certain embodiment, the cetyl dimethicone copolyol is Cetyl PEG/PPG-10/1 Dimethicone. According to additional embodiments, the volatile solvent is a non-polar volatile siloxane. According to some embodiments, the volatile siloxane is a linear polymethylsiloxane or a cyclic polymethylsiloxane. According to further embodiments, the volatile polymethylsiloxane is selected from the group consisting of hexamethyldisiloxane, heptamethyloctyltrisiloxane octamethylcyclotetrasiloxane, octamethyltrisiloxane, decamethylcyclopentasiloxane, decamethyltetrasiloxane, dodecamethylpentasiloxane, dodecamethylcyclohexasiloxane, and combinations thereof. According to a certain embodiment, the volatile polydimethylsiloxane is hexamethyldisiloxane. According to additional embodiments, the vasoconstrictor in the compositions of the present invention is selected from the group consisting of vasoconstrictors and combinations thereof.

According to further embodiments, the vasoconstrictor is selected from the group consisting of phenylephrine, phenylephrine hydrochloride, epinephrine, epinephrine hydrochloride, tetrahydrozoline hydrochloride, an amphetamine, an antihistamine, methylphenidate, mephedrone, oxymetazoline, pseudoephedrine, psilocybin, ephedrine sulfate, and combinations thereof. According to an exemplary embodiment, the vasoconstrictor is phenylephrine. According to some embodiments, the vasoconstrictor is present in the liquid adhesive composition in an amount ranging from about 0.005% to about 2% w/w.

According to some embodiments, the vasoconstrictor is present in the liquid adhesive composition in an amount ranging from about 0.01% to about 2% w/w. According to some embodiments, the vasoconstrictor is present in the liquid adhesive composition in an amount ranging from about 0.05% to about 2% w/w. According to some embodiments, the vasoconstrictor is present in the liquid adhesive composition in an amount ranging from about 0.1% to about 2% w/w. According to some embodiments, the vasoconstrictor is present in the liquid adhesive composition in an amount ranging from about 0.5% to about 2% w/w. According to some embodiments, the vasoconstrictor is present in the liquid adhesive composition in an amount ranging from about 1% to about 2% w/w. According to some embodiments, the vasoconstrictor is present in the liquid adhesive composition in an amount ranging from about 1.5% to about 2% w/w. According to some embodiments, the vasoconstrictor is present in the liquid adhesive composition in an amount ranging from about 0.005% to about 1.5% w/w. According to some embodiments, the vasoconstrictor is present in the liquid adhesive composition in an amount ranging from about 0.005% to about 1% w/w. According to some embodiments, the vasoconstrictor is present in the liquid adhesive composition in an amount ranging from about 0.005% to about 0.5% w/w. According to some embodiments, the vasoconstrictor is present in the liquid adhesive composition in an amount ranging from about 0.005% to about 0.1% w/w. According to some embodiments, the vasoconstrictor is present in the liquid adhesive composition in an amount ranging from about 0.005% to about 0.05% w/w. According to some embodiments, the vasoconstrictor is present in the liquid adhesive composition in an amount ranging from about 0.005% to about 0.01% w/w. According to some embodiments, the vasoconstrictor is present in the liquid adhesive composition in an amount ranging from about 0.5% to about 1.0% w/w. According to some embodiments, the vasoconstrictor is present in the liquid adhesive composition in an amount ranging from about 0.1% to about 1.0% w/w.

According to some embodiments, the vasoconstrictor is present in the liquid adhesive composition in an amount ranging from about 0.05% to about 20.0% w/w. According to some embodiments, the vasoconstrictor is present in the liquid adhesive composition in an amount ranging from about 0.1% to about 20.0% w/w. According to some embodiments, the vasoconstrictor is present in the liquid adhesive composition in an amount ranging from about 1.0% to about 20.0% w/w. According to some embodiments, the vasoconstrictor is present in the liquid adhesive composition in an amount ranging from about 5.0% to about 20.0% w/w. According to some embodiments, the vasoconstrictor is present in the liquid adhesive composition in an amount ranging from about 10.0% to about 20.0% w/w. According to some embodiments, the vasoconstrictor is present in the liquid adhesive composition in an amount ranging from about 15.0% to about 20.0% w/w. According to some embodiments, the vasoconstrictor is present in the liquid adhesive composition in an amount ranging from about 0.05% to about 15.0% w/w. According to some embodiments, the vasoconstrictor is present in the liquid adhesive composition in an amount ranging from about 0.05% to about 10.0% w/w. According to some embodiments, the vasoconstrictor is present in the liquid adhesive composition in an amount ranging from about 0.05% to about 5.0% w/w. According to some embodiments, the vasoconstrictor is present in the liquid adhesive composition in an amount ranging from about 5.0% to about 15.0% w/w. According to some embodiments, the vasoconstrictor is present in the liquid adhesive composition in an amount ranging from about 1.0% to about 10.0% w/w.

According to some embodiments, the pharmaceutical liquid adhesive composition can further comprise an additive selected from the group consisting of a dimethicone/vinyl dimethicone cross-polymer, a silicone gum blend, a gelling agent and a combination thereof. Each possibility is a separate embodiment of the invention.

According to a certain embodiment, the dimethicone/vinyl dimethicone cross-polymer comprises bis-vinyldimethicone, vinyldimethicone and hydrogen dimethicone.

According to additional embodiments, the silicone gum blend comprises a blend of high and low molecular weight silicones. According to a certain embodiment, the silicone gum blend comprises cyclopentasiloxane and dimethiconol.

According to some embodiments, the pharmaceutical liquid adhesive composition comprises: (i) trimethylsiloxysilicate; (ii) a surfactant selected from the group consisting of an anionic surfactant, a nonionic surfactant and a combination thereof; (iii) a volatile solvent selected from the group consisting of a polymethylsiloxane, an aliphatic hydrocarbon, and a combination thereof, (iv) water; and (v) at least one vasoconstrictor selected from the group consisting of vasoconstrictors. According to a certain embodiment, the surfactant is an anionic surfactant. According to some embodiments, the pharmaceutical liquid adhesive composition further comprises an additive selected from the group consisting of a dimethicone/vinyl dimethicone cross-polymer, a silicone gum blend, a gelling agent and a combination thereof.

According to some embodiments, the pharmaceutical liquid adhesive composition comprises: (i) about 10-40% w/w of trimethylsiloxysilicate; (ii) about 0.5-7% w/w of a surfactant selected from the group consisting of sodium lauryl sulfate, alkyl- and alkoxy-dimethicone copolyol, polysorbate, and a combination thereof; (iii) about 30-80% w/w of a volatile solvent, selected from the group consisting of polymethylsiloxane, a non-polar volatile siloxane and combinations thereof; (iv) about 20-40% w/w of water; and (v) about 0.005-25% w/w of at least one vasoconstrictor, selected from the group consisting of amphetamines, antihistamines, methylphenidate, mephedrone, oxymetazoline, phenylephrine, pseudoephedrine, psilocybin, phenylephrine hydrochloride, ephedrine sulfate, epinephrine, epinephrine hydrochloride, tetrahydrozoline hydrochloride, and combinations thereof. According to a certain embodiment, the at least one surfactant is sodium lauryl sulfate. According to another embodiment, the surfactant is a combination of sodium lauryl sulfate and cetyl dimethicone copolyol.

In some embodiments, the composition includes between 10-40% w/w of water. In some embodiments, the composition includes between 15-40% w/w of water. In some embodiments, the composition includes between 20-40% w/w of water. In some embodiments, the composition includes between 25-40% w/w of water. In some embodiments, the composition includes between 30-40% w/w of water. In some embodiments, the composition includes between 35-40% w/w of water. In some embodiments, the composition includes between 10-35% w/w of water. In some embodiments, the composition includes between 10-30% w/w of water. In some embodiments, the composition includes between 10-25% w/w of water. In some embodiments, the composition includes between 10-20% w/w of water. In some embodiments, the composition includes between 10-15% w/w of water. In some embodiments, the composition includes between 15-35% w/w of water. In some embodiments, the composition includes between 20-30% w/w of water.

According to additional embodiments, the surfactant is a combination of polysorbate and cetyl dimethicone copolyol.

According to some embodiments, cetyl dimethicone copolyol is Cetyl PEG/PPG-10/1 Dimethicone.

According to some embodiments, polymethylsiloxane is hexamethyldisiloxane. According to additional embodiments, volatile aliphatic hydrocarbon is isooctane.

According to some embodiments, the pharmaceutical liquid adhesive composition further comprises about 0.2-15% w/w of an additive selected from the group consisting of bis-vinyldimethicone, vinyldimethicone and hydrogen dimethicone; cyclopentasiloxane and dimethiconol and combinations thereof. According to some embodiments, the pharmaceutical liquid adhesive composition further comprises about 1-15% w/w of an additive selected from the group consisting of bis-vinyldimethicone, vinyldimethicone and hydrogen dimethicone; cyclopentasiloxane and dimethiconol and combinations thereof. According to some embodiments, the pharmaceutical liquid adhesive composition further comprises about 5-15% w/w of an additive selected from the group consisting of bis-vinyldimethicone, vinyldimethicone and hydrogen dimethicone; cyclopentasiloxane and dimethiconol and combinations thereof. According to some embodiments, the pharmaceutical liquid adhesive composition further comprises about 10-15% w/w of an additive selected from the group consisting of bis-vinyldimethicone, vinyldimethicone and hydrogen dimethicone; cyclopentasiloxane and dimethiconol and combinations thereof. According to some embodiments, the pharmaceutical liquid adhesive composition further comprises about 0.2-10% w/w of an additive selected from the group consisting of bis-vinyldimethicone, vinyldimethicone and hydrogen dimethicone; cyclopentasiloxane and dimethiconol and combinations thereof. According to some embodiments, the pharmaceutical liquid adhesive composition further comprises about 0.2-5% w/w of an additive selected from the group consisting of bis-vinyldimethicone, vinyldimethicone and hydrogen dimethicone; cyclopentasiloxane and dimethiconol and combinations thereof. According to some embodiments, the pharmaceutical liquid adhesive composition further comprises about 0.2-1% w/w of an additive selected from the group consisting of bis-vinyldimethicone, vinyldimethicone and hydrogen dimethicone; cyclopentasiloxane and dimethiconol and combinations thereof. According to some embodiments, bis-vinyldimethicone, vinyldimethicone and hydrogen dimethicone can be present in the liquid adhesive composition in an amount ranging from about 5 to 15% w/w.

According to further embodiments, cyclopentasiloxane and dimethiconol can be present in the liquid adhesive composition in an amount ranging from about 0.5 to 2.5% w/w.

According to some embodiments, the pharmaceutical liquid adhesive composition comprises: (i) about 15% w/w trimethylsiloxysilicate; (ii) about 1.5% w/w sodium lauryl sulfate; (iii) about 22% w/w hexamethyldisiloxane and 21% w/w isooctane; (iv) about 25% w/w water about 12% of a silicone acrylate and (v) about 0.05% w/w phenylephrine as the vasoconstrictor.

According to a certain embodiment, the pharmaceutical liquid adhesive composition comprises: (i) about 15% w/w silicone acrylate (ii) about 1.5% w/w sodium lauryl sulfate; (iii) about 22% w/w hexamethyldisiloxane and 21% w/w isooctane; (iv) about 26% w/w water and (vi) about 0.05% w/w phenylephrine.

According to further embodiments, the pharmaceutical liquid adhesive composition comprises: (i) about 15% w/w trimethylsiloxysilicate; (ii) about 1.5% w/w sodium lauryl sulfate; (iii) about 4% w/w Cetyl PEG/PPG-10/1 Dimethicone (iv) about 22% w/w hexamethyldisiloxane and 21% w/w isooctane; (v) about 25% w/w water; and (vi) about 1% w/w oxymethazoline as the vasoconstrictor and about 12% of a silicone acrylate. Alternatively, the vasoconstrictor is phenylephrine in an amount of about 0.05% w/w. Further alternatively, the vasoconstrictor is a combination of about 1% w/w oxymethazoline and about 0.05% w/w phenylephrine.

According to still further embodiments, the pharmaceutical liquid adhesive composition comprises: (i) about 15% w/w trimethylsiloxysilicate; (ii) about 1.5% w/w Tween 80; (iii) about 4% w/w Cetyl PEG/PPG-10/1 Dimethicone (iv) about 22% w/w hexamethyldisiloxane and 21% w/w isooctane; (v) about 25% w/w water about 12% of a silicone acrylate and (vi) about 1% w/w oxymethazoline as the vasoconstrictor. Alternatively, the vasoconstrictor is phenylephrine in an amount of about 0.05% w/w. Further alternatively, the vasoconstrictor is a combination of about 1% w/w oxymethazoline and about 0.05% w/w phenylephrine.

According to some embodiments, the pH of the liquid adhesive composition is from about 3.5 to about 5. According to other embodiments, the pH of the liquid adhesive composition is from about 4.0 to about 4.6. According to additional embodiments, the pH of the liquid adhesive composition is from about 4.2 to about 4.4.

According to another aspect, the present invention provides a method of treating or preventing an Epistaxis disorder, the method comprising the step of topically applying to a mucosal surface of the nose, in the affected nostril of a subject in need of such treatment a therapeutically effective amount of the pharmaceutical liquid adhesive composition of the present invention.

According to one embodiment, the subject to be treated is a human being. According to another embodiment, the subject to be treated is an animal.

According to yet another aspect, the present invention provides a kit comprising a pharmaceutical liquid adhesive composition and a container-applicator device suitable for storage and application of the composition to the nose, in the affected nostril.

According to some embodiments, the container-applicator device is selected from the group consisting of a single use wipe, a syringe, a dropper, a spray dispenser, a swab, a compressible bottle or tube, a spatula, a suppository insertion tube, an extrusion tube and an inflatable member.

According to another aspect, the present invention provides a pharmaceutical liquid adhesive composition for use in treating or preventing an Epistaxis disorder.

Other objects, features and advantages of the present invention will become clear from the following description and claims.

### Definitions

The wording herein below is implied in the common meaning of the definitions and statements as known to the versed in the art of pharmaceuticals and polymer science. However, there are several terms that should be understood in the concept of present invention as follows:

As used in the specification and claims, the forms “a”, “an” and “the” include singular as well as plural references unless the context clearly dictates otherwise.

Further, as used herein, the term “comprising” is intended to mean that the system includes the recited elements, but not excluding others which may be optional in the design of the system, such as fillers and the like. The term “consisting essentially of” is used to define a system that includes the recited elements but exclude other elements that may have an essential significance effect on the performance of the system. “Consisting of” shall thus mean excluding more than traces of other elements. Embodiments defined by each of these transition terms are within the scope of this invention.

The term “film forming agent” or “film former”, as used herein, means a silicone resin that after dissolution in at least one solvent leaves a film on the substrate to which it is applied, for example once the at least one solvent evaporates, absorbs and/or dissipates on the substrate.

The term “volatile solvent”, as used herein, means that the solvent has a measurable vapor pressure.

### DETAILED DESCRIPTION OF THE INVENTION

### Compositions for the Treatment of Epistaxis

The liquid adhesive compositions of the present invention comprise a silicon resin film forming agent, at least one surfactant, a volatile solvent, water, optionally a silicone acrylate and at least one pharmaceutically active agent. The liquid adhesive composition can further comprise additives, such as dimethicone/vinyl dimethicone cross-polymers, silicone gum blends and gelling agents.

The silicone resin according to the present invention is a film forming agent. The non-limiting examples of silicone resins useful in the compositions of the invention are silicone acrylates, siloxysilicates, silsesquioxanes (usually denoted as T-resins) and a combination thereof One non-limiting example of a siloxysilicate in accordance with the present invention is trimethylsiloxysilicate, which may be represented by the following formula:

[(CH3)3—Si—O]x—(SiO4/2)y

wherein x and y may, for example, range from 50 to 80. Such siloxysilicates are commercially available from General Electric and Dow Corning under the trade name Resin MQ®. One non-limiting example of silsesquioxane is polymethylsilsesquioxane. The present invention discloses the use of trimethylsiloxysilicate for therapeutic applications, inter alia, for treatment of Epistaxis. Trimethylsiloxysilicate is soluble in the volatile solvent of the liquid adhesive composition. The amount of the silicon resin film forming agent in the composition is determined based on the desired adhesion properties of the film to the target surface. The amount depends, inter alia, on the target surface, the condition to be treated, and the amount of composition ingredients. The amount of the silicone resin film forming agent further defines the viscosity of the liquid adhesive composition. The amount of the silicone resin film forming agent in the composition ranges from about 10% to 40% w/w. The term “about” as used herein denotes ±10% of the value indicated.

The volatile solvent useful for dissolving the silicone resin is chosen from volatile silicon or volatile aliphatic hydrocarbon. Water solubility of the volatile solvent is less than about 0.1%. According to some embodiments, the volatile silicone is a linear or cyclic polymethylsiloxane, having from 2 to 9 silicon atoms, these silicones being optionally substituted with alkyl or alkoxy groups of 1 to 10 carbon atoms. The non-limiting examples of polydimethylsiloxanes in accordance with the present invention are hexamethyldisiloxane, heptamethyloctyltrisiloxane octamethylcyclotetrasiloxane, octamethyltrisiloxane, decamethylcyclopentasiloxane, decamethyltetrasiloxane, dodecamethylpentasiloxane, dodecamethylcyclohexasiloxane, and mixtures thereof. The polydimethylsiloxane used in the compositions is hexamethyldisiloxane.

The volatile solvent can further comprise a volatile aliphatic hydrocarbon. The aliphatic hydrocarbon in accordance with the present invention may be any aliphatic hydrocarbon, including an alkane, a mixture of alkanes, an alkene, a mixture of alkenes, an alkyne, a mixture of alkynes, an ester or a mixture thereof. The aliphatic hydrocarbon is an alkane such as pentane, isooctane, isododecane, isohexadecane or a mixture thereof. According to a certain embodiment, the aliphatic hydrocarbon is isooctane. The volatile ester useful for dissolving the film former may be a branched ester, such as isohexyl or isodecyl neopentanoate and mixture thereof.

The volatile solvent may comprise a volatile silicone, a volatile aliphatic hydrocarbon or a mixture thereof. According to a certain embodiment, the volatile solvent comprises hexamethyldisiloxane and isooctane.

According to some embodiments, the water presence in the liquid adhesive composition of the present invention allows dissolution of the pharmaceutically active agents, which are not soluble in the non-polar volatile solvents used for dissolving the film-former. The emulsion can be a water-in-oil or oil-in-water emulsion. According to exemplary embodiments, the liquid adhesive composition of the present invention is an oil-in-water emulsion, wherein the aqueous phase includes the vasoconstrictors dissolved therein and the oil phase includes the film forming agent dissolved in the volatile solvent. The oil-in-water emulsion allows the film former and the pharmaceutical active agents to be homogeneously dispersed in the liquid adhesive composition. The stable emulsion provides fine dispersion of the emulsion ingredients in the liquid adhesive composition, in the container-applicator device and upon the application to the target surface, such that once the volatile solvent and water evaporate both the film former and the pharmaceutical active ingredients remain finely dispersed on the target surface. The stable emulsion prevents clamping, floating and/or precipitation of the polar active ingredients in the non-polar volatile solvents. The presence of the aqueous phase in the liquid adhesive composition further obviates the use of polar solvents, formerly required to dissolve and disperse pharmaceutical active ingredients in silicone based liquid bandages.

The amount of the volatile solvent and water affects the viscosity and evaporation time of the liquid adhesive composition when applied to a target surface. The amount of the volatile solvent and water can be determined by a person skilled in art so as to adjust the viscosity and evaporation time to desired values. The amount of volatile solvent and water further affects the morphology of the silicone/water emulsion. The amount of the volatile solvent can be adjusted to obtain the desired emulsion type. The amount of the volatile solvent in the composition ranges from about 30% to about 80% w/w. The amount of water can be adjusted to obtain the desired emulsion type. The amount of water in the composition ranges from about 10% to about 40% w/w.

The liquid adhesive composition further comprises at least one surfactant. Addition of the surfactant allows mixing of the silicone and the aqueous phases, producing a silicone/water emulsion. Addition of the surfactant further allows the emulsion stabilization. As described hereinabove, the obtained emulsion may be an oil-in-water emulsion, wherein the aqueous phase includes dissolved pharmaceutical ingredients and finely dispersed volatile solvent phase, containing the dissolved film former.

The surfactant is selected from the group consisting of an anionic surfactant, a non-ionic surfactant, selected from organosilicon surfactant or nonionic organic surfactant, a cationic surfactant, an amphoteric surfactant and a combination thereof. Each possibility is a separate embodiment of the invention.

The anionic surfactants usable in the compositions of the present invention include sodium alkyl sulfates, such as, but not limited to sodium lauryl sulfate; sodium alkyl sulfonates; sodium alkyl aryl sulfonates, such as sodium dodecyl benzene sulfonate and the like; sodium stearate; dioctyl sodium sulfosuccinate; sodium cholate; and a combination thereof.

Examples of suitable organosilicon surfactants include, but are not limited to dimethicone copolyols such as: alkoxy dimethicone copolyols, alkyl and alkoxy-dimethicone copolyols, silicones having pendant hydrophilic moieties such as linear silicones having pendant polyether groups, branched polyether and alkyl modified silicones, branched polyglycerin and alkyl modified silicones. In some embodiments, the dimethicone copolyol is cetyl dimethicone copolyol, such as Cetyl PEG/PPG-10/1 Dimethicone sold under the name Abil EM-90. Other suitable dimethicone copolyols include branched polyether and alkyl modified silicones such as Lauryl PEG-9 Polydimethylsiloxyethyl Dimethicone sold under the name KF-6038, and branched polyglycerin and alkyl modified silicones such as Lauryl Polyglyceryl-3 Polydimethylsiloxyethyl dimethicone sold under the name KF-6105. Additional dimethicone copolyols useful in the compositions of the present invention include bis-PEG/PPG-14/dimethicone copolyol sold under the name Abil EM-97 and the polyglyceryl-4 isostearate/cetyl dimethicone copolyol/hexyl laurate mixture sold under the name Abil WE 09. Another suitable dimethicone copolyol is PEG-9 Polydimethylsiloxyethyl Dimethicone sold under the name KF-6028. Abil EM-90, Abil EM-97 and Abil WE 09 are available from Evonik Goldschmidt GmbH of Essen, Germany. KF-6038 are KF-6105 are available from Shin-Etsu Silicones of Akron, Ohio.

Non-limiting examples of possible non-ionic organic surfactants include polysorbates, such as polyoxyethylene sorbitan monolaurate (Tween 20), polyoxyethylene sorbitan monopalmitate (Tween 40), polyoxyethylene sorbitan monostearate (Tween 60) and polyoxyethylene sorbitan monooleate (Tween 80); glyceryl stearate; polyoxyethylene (POE) fatty acid esters, such as Myrj 45, Myrj 49, Myrj 52 and Myrj 59; poly(oxyethylene) alkylyl ethers, such as poly(oxyethylene) cetyl ether (Brij 52, Brij 56, Brij 58), poly(oxyethylene) palmityl ether, polyethylene oxide hexadecyl ether, polyethylene glycol cetyl ether, and the like; polyethoxylene castor oil derivatives, such as Cremophor EL, ELP and RH 40; PEG-6 octanoic/decanoic glycerides, such as Softigen 767 and the like; polyoxyethylene glycerol trioleate, such as but not limited to Tagat TO; decaglycerol mono/dioleate, such as Caprol PGE860 and the like; and a combination thereof.

The nonionic organic surfactants may further comprise sorbitan fatty acid esters, such as sorbitan monolaurate (Span 20), sorbitan monopalmitate (Span 40), sorbitan monooleate (Span 80), sorbitan monostearate (Span 60); mono/diglycerides of octanoic/decanoic acids, such as but not limited to Imwitor-742, Imwitor-308, and a combination thereof.

Non-limiting examples of possible cationic surfactants include phosphatides, such as phosphatidyl choline and the like; quaternary ammonium cationic surfactants, such as hexadecyltrimethyl ammonium bromide and the like; pyrimidinium cationic surfactants, such as, but not limited to dodecyl pyridinium chloride; and a combination thereof.

The amphoteric surfactant may include lecithine, N-dodecyl alanine, cocamidopropyl amino betaine or a combination thereof.

The type and the amount of surfactant may be determined by a person skilled in art so as to obtain the Hydrophile-Liphophile Balance (HLB) of the surfactant or the surfactant mixture suitable for the oil-in-water systems.

According to some embodiments, the surfactant used in the compositions of the present invention is an anionic surfactant. According to additional embodiments, the surfactant may further comprise nonionic surfactant. The nonionic surfactant may be selected from the group consisting of nonionic organic surfactant, organosilicon surfactant and a combination thereof. According to other embodiments, the surfactant in the compositions of the present invention is a nonionic surfactant.

In some embodiments, the surfactant is sodium alkyl sulfate, such as sodium lauryl sulfate. According to other embodiments, the surfactant is a combination of sodium alkyl sulfate and alkyl and alkoxy-dimethicone copolyol, for example, sodium lauryl sulfate and Cetyl PEG/PPG-10/1 Dimethicone. According to other embodiments, the surfactant is selected from polyoxyethylene sorbitan monolaurate (Tween 20), polyoxyethylene sorbitan monopalmitate (Tween 40), polyoxyethylene sorbitan monostearate (Tween 60), polyoxyethylene sorbitan monooleate (Tween 80) or any mixture thereof. According to further embodiments, the silicone surfactant is a combination of polysorbate alkyl and alkoxy-dimethicone copolyol, for example, polyoxyethylene sorbitan monooleate (Tween 80) and Cetyl PEG/PPG-10/1 Dimethicone.

In some embodiments, the composition of the present invention includes between 0.1-10% w/w of a surfactant. In some embodiments, the composition of the present invention includes between 0.5-10% w/w of a surfactant. In some embodiments, the composition of the present invention includes between 1-10% w/w of a surfactant. In some embodiments, the composition of the present invention includes between 5-10% w/w of a surfactant. In some embodiments, the composition of the present invention includes between 0.1-5% w/w of a surfactant. In some embodiments, the composition of the present invention includes between 0.1-1% w/w of a surfactant. In some embodiments, the composition of the present invention includes between 0.1-0.5% w/w of a surfactant. In some embodiments, the composition of the present invention includes between 0.5-5% w/w of a surfactant.

The liquid adhesive composition of the present invention may further comprise an additive selected from the group consisting of dimethicone/vinyldimethicone cross-polymers, silicone gum blends, gelling agents, and a combination thereof.

The dimethicone/vinyldimethicone cross-polymer is available, for example, from Dow Corning as Dow Corning 9506 Cosmetic Powder. According to other embodiments, the dimethicone/vinyldimethicone cross-polymer can be present in the compositions of the present invention in a form of two-part silicone elastomer. Without being bound to any mechanism of action, the addition of two-part silicone elastomers to the liquid adhesive composition can provide enhanced film adhesion onto the target surface and can allow reduction of skin strain, which may be caused by the silicone resin. The two-part silicone elastomers form a cross-polymer network by addition reaction, upon mixing the two parts, enhancing the composition adhesive properties. One part of the two-part silicone elastomer usually contains vinyl end-blocked silicone polymer and a catalyst suitable for promoting the addition reaction and another part contains vinyl end-blocked silicone polymer and silicone polymer carrying SiH groups. These two parts are stored separately before use and the cross-linking reaction starts upon mixing the two parts in a defined ratio. The ratio of the two parts is usually 50:50 and the cross-linking reaction may proceed at room temperature (25±5° C.). The two-part silicone elastomers may comprise dimethicone, hydrogen dimethicone, vinyldimethicone, bis-vinyldimethicon and phenyltrimethicone. According to a certain embodiment, the liquid adhesive composition of the present invention comprises bis-vinyldimethicone as the first part of the two-part silicone elastomers and vinyldimethicone and hydrogen dimethicone as the second part. The first part can further contain a platinum catalyst. The bis-vinyldimethicone, vinyldimethicone and hydrogen dimethicone are available, for example, from KCC as SM9010™ or SM9020™. The amount of the dimethicone/vinyldimethicone in the composition may be in a range from about 5% to 15% w/w.

In some embodiments, the liquid adhesive may further comprise a silicone gum blend. Without being bound to any mechanism of action, the addition of the silicone gum blend provides enhancement of silkiness of the film. Silicone gum blend may be a blend of a high molecular weight and a low molecular weight silicone. The average molecular weight of the high molecular weight silicone is 100,000 or greater. The average molecular weight of the low molecular weight silicone is 10,000 or less. High molecular and low molecular weight silicones may comprise dimethicone and/or dimethiconol. The non-limiting examples of a silicone gum blend are cyclopentasiloxane and dimethiconol, and cyclotetrasiloxane and cyclopentasiloxane and dimethiconol. The cyclopentasiloxane and dimethiconol blends are available, for example, from KCC as SF9902E™ or from Momentive as Silsoft 1215 dimethicone™. The amount of the silicone gum blend in the composition may be in a range from about 0.5% to 2.5% w/w. The amount of the silicone gum blend in the composition may be in a range from about 0.5% to 2.0% w/w. The amount of the silicone gum blend in the composition may be in a range from about 0.5% to 1.5% w/w. The amount of the silicone gum blend in the composition may be in a range from about 0.5% to 1.0% w/w. The amount of the silicone gum blend in the composition may be in a range from about 1.0% to 2.5% w/w. The amount of the silicone gum blend in the composition may be in a range from about 1.5% to 2.5% w/w. The amount of the silicone gum blend in the composition may be in a range from about 2.0% to 2.5% w/w.

In some embodiments of the composition of the present application, upon application of the liquid adhesive to the target surface, the volatile solvent and water evaporate, leaving an adhered film which includes at least one pharmaceutically active agent. It is to be appreciated that the compositions of the present invention are devoid of polar solvents required for dissolving active ingredients, thus providing non-stinging liquid adhesives that have a comfortable feel when applying in the nostril.

### Pharmaceutical Active Agents

The liquid adhesive compositions of the present invention comprise at least one pharmaceutically active agent, such as a vasoconstrictor. The compositions may further contain one or more protectant active ingredients, excipients and carriers. Pharmaceutically and dermatologically acceptable excipients and carriers as are known in the art may be included in the composition, in particular for maintaining the stability and sterility of the composition, and for promoting delivery, release and/or application of the active agent(s) to the body surface to which the composition is applied.

It is to be understood that the compositions may contain more than one active agent, and/or may be suitable for use in treating different Epistaxis disorders. The pharmaceutically active agent and the dosage thereof is dependent upon the particular condition to be treated, the age of the subject and other factors evident to those skilled in the art. Vasoconstrictors which are suitable for use in the invention include amphetamines, antihistamines, methylphenidate, mephedrone, oxymetazoline, phenylephrine, pseudoephedrine, psilocybin, phenylephrine hydrochloride, ephedrine sulfate, epinephrine, epinephrine hydrochloride, tetrahydrozoline hydrochloride, and combinations thereof. Suitable amounts of such vasoconstrictor agents in the composition may be readily ascertained by one of ordinary skill in the art, and may range, for example, between 0.005% and 2% w/w. Exemplary vasoconstrictor agent is phenylephrine HCl. In a particular embodiment, the composition of the invention comprises phenylephrine HCl at a concentration of about 0.05% w/w based on the total weight of the composition.

Antifibrinolytics which are suitable for use in the invention include tranexamic acid, aprotinin, ε-aminocaproic acid, aminomethylbenzoic acid or their salts and combinations thereof.

The compositions may further include protectant active ingredients. The protectant active ingredients can be selected from the group consisting of aluminum hydroxide gel, cocoa butter, aqueous solution of glycerin, hard fat, kaolin, lanolin, mineral oil, petrolatum, topical starch, white petrolatum, cod liver, shark liver oil, and a combination thereof. The protectant active ingredient and the dosage thereof is dependent upon the particular condition to be treated, the pharmaceutical active agents present in the composition and other factors evident to those skilled in the art.

The present composition may include one or more of the following additional ingredients: emulsifiers (e.g. anionic, cationic or nonionic), chelating agents, colorants, emollients, fragrances, humectants, lubricants, moisturizers, preservatives, skin penetration enhancers, stabilizers, thickeners, and viscosity modifiers.

### Silicone Acrylates

Silicone acrylates are a novel and important ingredient of the compositions of the instant invention. Silicone acrylates can provide better compatibility with organic compounds. They are good film formers and have good compatibility and capacity with w/o siliconic surfactants. We believe silicon acrylates were not used before for this purpose.

Throughout this application, the term “silicone acrylate” is meant as acrylates/polytrimethyl-siloxymethacrylate copolymers generically designated as “silicone acrylate”.

Non-limiting examples of silicone acrylates are Dow Corning® FA 4001 CM Silicone Acrylate (copolymer of cyclopentasiloxane and acrylates/polytrimethylsiloxymethacrylate) and Dow Corning® FA 4002 CM Silicone Acrylate (copolymer of acrylates/polytrimethylsiloxymethacrylate in isododecane).

In an embodiment, there are provided liquid adhesive compositions comprising between 1-20% silicon acrylates.

In another embodiment, there are provided liquid adhesive compositions comprising between 1-20% silicone acrylates, 0-3% sodium alkyl sulfate, 30-70% volatile solvent, 0-40% water, 2-10% silicone surfactant and 0.05-5% of at least one pharmaceutical.

In another embodiment, there are provided liquid adhesive compositions comprising between 10-15% silicone acrylates, 0-3% sodium alkyl sulfate, 50-70% volatile solvent, 10-30% water, 4-7% silicone surfactant and 0.05-5% of at least one pharmaceutical.

There are provided pharmaceutical liquid adhesive compositions comprising:

(i) a silicon acrylate

(ii) at least one surfactant;

(iii) a volatile solvent;

(iv) water; and

(v) at least one vasoconstrictor,
wherein the composition is in the form of an emulsion.

The pharmaceutical liquid adhesive emulsions, of the instant invention may be water-in-oil emulsions.

The pharmaceutical liquid adhesive compositions comprise at least one surfactant wherein the at least one surfactant is a siliconic surfactant and the optional additional surfactant is anionic.

The optional additional anionic surfactant above in the pharmaceutical liquid adhesive compositions is selected from the group consisting of sodium alkyl sulfate, sodium alkyl sulfonate, sodium alkyl aryl sulfonate, sodium stearate, dioctyl sodium sulfosuccinate, sodium cholate, and any combination thereof.

The sodium alkyl sulfate in the pharmaceutical liquid adhesive compositions, may be sodium lauryl sulfate.

The at least one surfactant in the pharmaceutical liquid adhesive compositions may be a nonionic surfactant.

The nonionic surfactant in the pharmaceutical liquid adhesive compositions according is selected from the group consisting of organosilicon surfactants, nonionic organic surfactants, and combinations thereof.

The organosilicon surfactant in the pharmaceutical liquid adhesive composition is selected from alkyl- and alkoxy-dimethicone copolyol.

The alkyl- and alkoxy-dimethicone copolyol in the pharmaceutical liquid adhesive compositions may be cetyl dimethicone copolyol.

The cetyl dimethicone copolyol in the pharmaceutical liquid adhesive compositions according may be Cetyl PEG/PPG-10/1 Dimethicone.

The nonionic organic surfactant in the pharmaceutical liquid adhesive compositions is selected from the group consisting of polysorbate, glyceryl stearate, polyoxyethylene (POE) fatty acid ester, poly(oxyethylene) alkylyl ether, polyethoxylene castor oil derivative, PEG-6 octanoic/decanoic glycerides, polyoxyethylene glycerol trioleate, decaglycerol mono/dioleate, and any combination thereof.

The polysorbate in the pharmaceutical liquid adhesive composition is selected from the group consisting of polyoxyethylene sorbitan monolaurate (Tween 20), polyoxyethylene sorbitan monopalmitate (Tween 40), polyoxyethylene sorbitan monostearate (Tween 60) and polyoxyethylene sorbitan monooleate (Tween 80).

The at least one surfactant in the pharmaceutical liquid adhesive composition is selected from the group consisting of a cationic surfactant, an amphoteric surfactant, and a combination thereof.

The volatile solvent in the pharmaceutical liquid adhesive compositions is selected from the group consisting of a volatile polymethylsiloxane, a volatile aliphatic hydrocarbon, and combinations thereof.

The volatile polydimethylsiloxane in the pharmaceutical liquid adhesive composition is selected from the group consisting of hexamethyldisiloxane, heptamethyloctyltrisiloxane, octamethylcyclotetrasiloxane, octamethyltrisiloxane, decamethylcyclopentasiloxane, decamethyltetrasiloxane, dodecamethylpentasiloxane, dodecamethylcyclohexasiloxane, and a combination thereof.

The volatile aliphatic hydrocarbon in the pharmaceutical liquid adhesive composition is selected from the group consisting of alkanes, alkenes, alkynes, and a combination thereof.

The alkane in the pharmaceutical liquid adhesive composition is selected from the group consisting of pentane, isooctane, isododecane, isohexadecane and a combination thereof.

The vasoconstrictor in the pharmaceutical liquid adhesive composition is selected from the group consisting of vasoconstrictors, and combinations thereof.

The vasoconstrictor in the pharmaceutical liquid adhesive composition is selected from the group consisting of phenylephrine, phenylephrine hydrochloride, epinephrine, epinephrine hydrochloride, tetrahydrozoline hydrochloride, an amphetamine, an antihistamine, methylphenidate, mephedrone, oxymetazoline, pseudoephedrine, psilocybin, ephedrine sulfate, and combinations thereof.

The vasoconstrictor in the pharmaceutical liquid adhesive composition is phenylephrine. There are provided pharmaceutical liquid adhesive compositions comprising:
(i) a silicone acrylate;
(ii) a surfactant selected from the group consisting of siliconic surfactants, anionic surfactants, nonionic surfactants, and combinations thereof;
(iii) a volatile solvent selected from the group consisting of a non-polar volatile siloxane, an aliphatic hydrocarbon, and combinations thereof;
(iv) water; and
(v) a vasoconstrictor

There are provided pharmaceutical liquid adhesive compositions comprising:
(i) about 10-40% w/w of trimethylsiloxysilicate;
(ii) about 0.5-7% w/w of a surfactant selected from the group consisting of sodium lauryl sulfate, alkyl- and alkoxy-dimethicone copolyol polysorbate and a combination thereof;
(iii) about 30-80% w/w of a volatile solvent selected from the group consisting of hexamethyldisiloxane, isooctane and combinations thereof;
(iv) about 20-40% w/w of water; and
(v) about 0.005-25% w/w of a vasoconstrictor selected from the group consisting of phenylephrine, phenylephrine hydrochloride, epinephrine, epinephrine hydrochloride, tetrahydrozoline hydrochloride, an amphetamine, an antihistamine, methylphenidate, mephedrone, oxymetazoline, pseudoephedrine, psilocybin, ephedrine sulfate, and combinations thereof.

In an embodiment, there are provided pharmaceutical liquid adhesive compositions comprising:
a. about 15-20% w/w trimethylsiloxysilicate;
b. about 1.5-3.0% w/w sodium lauryl sulfate;
c. about 22-30% w/w hexamethyldisiloxane and 20-25% w/w isooctane;
d. about 25-30% w/w water;
e. about 10-15% of a silicone acrylate
f. about 0.05-0.2% w/w phenylephrine.

Epistaxis is caused by one or more of the following causes: injury to the nose, hemophilia, upper respiratory infection, hypertension, antiplatelet medication, foreign body, insufflated drugs, barotrauma, nasal surgery, nasal sprays, allergic reactions or combinations thereof

There is provided a method of preventing or treating an Epistaxis disorder, comprising the step of topically applying to the mucosal surface of the affected nostril of a subject in need of such treatment a therapeutically effective amount of the liquid adhesive composition of this invention.

There is provided a pharmaceutical liquid adhesive composition for the treatment of any type of nose bleeding, including Epistaxis.

There is provided a kit comprising a pharmaceutical liquid adhesive composition and a container-applicator device suitable for storage and application of the emulsion to the nose, into the affected nostril.

There is provided a kit, wherein the container-applicator device comprises at least one of a single use wipe, a towelette, a syringe, a dropper, a spray dispenser, a compressible bottle or tube, a spatula, a suppository insertion tube, an extrusion tube, and an inflatable member and instructions for use.

There are provided pharmaceutical liquid adhesive compositions according to the instant invention, for use in preventing or treating an Epistaxis disorder.

According to some embodiments, the pharmaceutical liquid adhesive composition comprises: (i) a silicone resin film forming agent comprising (i) a silicone acrylate, siloxysilicate, silsesquioxane, or a derivative or a combination thereof; (ii) an anionic surfactant; (iii) a volatile solvent, (iv) water; and (v) at least one vasoconstrictor.

According to some embodiments, the pharmaceutical liquid adhesive compositions of this disclosure may be administered as gel, nasal swabs/wipes and nasal aerosols.

According to other embodiments, the pharmaceutical liquid adhesive composition comprises: (i) a silicone resin film forming agent comprising a silicone acrylate, siloxysilicate, silsesquioxane, or a derivative or a combination thereof; (ii) an anionic surfactant; (iii) a nonionic surfactant, (iv) a volatile solvent, (v) water; and (vi) at least one vasoconstrictor. According to additional embodiments, the pharmaceutical liquid adhesive composition comprises: (i) a silicone resin film forming agent comprising siloxysilicate, silsesquioxane, or a derivative or a combination thereof; (ii) a nonionic surfactant; (iii) a volatile solvent, (iv) water; and (v) at least one vasoconstrictor. According to further embodiments, the pharmaceutical liquid adhesive composition comprises: (i) a silicone resin film forming agent comprising a silicone acrylate, siloxysilicate, silsesquioxane, or a derivative or a combination thereof; (ii) an anionic surfactant; (iii) a volatile solvent, (iv) water; (v; and (vi) at least one vasoconstrictor. According to other embodiments, the pharmaceutical liquid adhesive composition comprises: (i) a silicone resin film forming agent comprising a silicone acrylate, siloxysilicate, silsesquioxane, or a derivative or a combination thereof; (ii) an anionic surfactant; (iii) a nonionic surfactant, (iv) a volatile solvent, (v) water; (vi) and (vii) at least one vasoconstrictor. According to additional embodiments, the pharmaceutical liquid adhesive composition comprises: (i) a silicone resin film forming agent comprising siloxysilicate, silsesquioxane, or a derivative or a combination thereof; (ii) a nonionic surfactant; (iii) a volatile solvent, (iv) water; (v); and (vi) at least one vasoconstrictor.

According to some embodiments, the pharmaceutical liquid adhesive composition comprises: (i) trimethylsiloxysilicate; (ii) sodium alkyl sulfate; (iii) a volatile solvent, selected from the group consisting of polydimethylsiloxane, aliphatic hydrocarbon and a combination thereof; (iv) water; and (v) at least one vasoconstrictor. According to certain embodiments, the pharmaceutical liquid adhesive composition comprises: (i) trimethylsiloxysilicate; (ii) sodium lauryl sulfate; (iii) a volatile solvent, selected from the group consisting of hexamethyldisiloxane, isooctane and a combination thereof; (iv) water; and (v) at least one vasoconstrictor.

According to other embodiments, the pharmaceutical liquid adhesive composition comprises: (i) trimethylsiloxysilicate; (ii) sodium alkyl sulfate; (iii) alkyl- and alkoxy-dimethicone copolyol; (iv) a volatile solvent, selected from the group consisting of polydimethylsiloxane, aliphatic hydrocarbon and a combination thereof; (v) water, (vi) a silicone acrylate and (vii) at least one vasoconstrictor. According to certain embodiments, the pharmaceutical liquid adhesive composition comprises: (i) trimethylsiloxysilicate; (ii) sodium lauryl sulfate; (iii) Cetyl PEG/PPG-10/1 Dimethicone; (iv) a volatile solvent, selected from the group consisting of hexamethyldisiloxane, isooctane, a silicone acrylate and a combination thereof; (v) water; and (vi) at least one vasoconstrictor.

According to other embodiments, the pharmaceutical liquid adhesive composition comprises: (i) trimethylsiloxysilicate; (ii) polysorbate; (iii) alkyl- and alkoxy-dimethicone copolyol; (iv) a volatile solvent, selected from the group consisting of polymethylsiloxanes, volatile aliphatic hydrocarbons and combinations thereof; (v) water (vi) at least one vasoconstrictor and (vii) a silicone acrylate. According to further embodiments, the pharmaceutical liquid adhesive composition comprises: (i) trimethylsiloxysilicate; (ii) Tween 80; (iii) Cetyl PEG/PPG-10/1 Dimethicone; (iv) a volatile solvent, selected from the group consisting of hexamethyldisiloxane, isooctane and a combination thereof; (v) water (vi) at least one vasoconstrictor and a silicone acrylate.

According to some embodiments, the pharmaceutical liquid adhesive composition comprises: (i) trimethylsiloxysilicate; (ii) sodium alkyl sulfate; (iii) a volatile solvent, selected from the group consisting of polydimethylsiloxane, aliphatic hydrocarbon and a combination thereof; (iv) water; (v) silicone acrylates and (vi) at least one vasoconstrictor. According to certain embodiments, the pharmaceutical liquid adhesive composition comprises: (i) trimethylsiloxysilicate; (ii) sodium lauryl sulfate; (iii) a volatile solvent, selected from the group consisting of hexamethyldisiloxane, isooctane and a combination thereof; (iv) water; (v) (vi) at least one vasoconstrictor and a silicone acrylate.

According to further embodiments, the pharmaceutical liquid adhesive composition comprises: (i) a silicone resin film forming agent comprising siloxysilicate, silsesquioxane, or a derivative or a combination thereof; (ii) a surfactant; (iii) a volatile solvent, (iv) water; (v) at least one vasoconstrictor; (vi) a dimethicone/vinyldimethicone cross-polymer; and (vii) a silicone gum blend. According to still further embodiments, the pharmaceutical liquid adhesive composition comprises: (i) a silicone resin film forming agent comprising siloxysilicate, silsesquioxane, or a derivative or a combination thereof; (ii) a surfactant; (iii) a volatile solvent, (iv) water; (v) at least one vasoconstrictor; (vi) a dimethicone/vinyldimethicone cross-polymer; (vii) a silicone gum blend; and (ix) a silicone acrylate. According to further embodiments, the pharmaceutical liquid adhesive composition comprises: (i) a silicone resin film forming agent comprising siloxysilicate, silsesquioxane, or a derivative or a combination thereof; (ii) anionic surfactant; (iii) a volatile solvent, (iv) water; (v) at least one vasoconstrictor; (vi) a dimethicone/vinyldimethicone cross-polymer; and (vii) a silicone gum blend. According to still further embodiments, the pharmaceutical liquid adhesive composition comprises: (i) a silicone resin film forming agent comprising siloxysilicate, silsesquioxane, or a derivative or a combination thereof; (ii) anionic surfactant; (iii) a volatile solvent, (iv) water; (v) at least one vasoconstrictor; (vi) a dimethicone/vinyldimethicone cross-polymer; (vii) a silicone gum blend; and (ix) a silicone acrylate.

According to further embodiments, the pharmaceutical liquid adhesive composition comprises: (i) a silicone resin film forming agent; (ii) anionic surfactant; (iii) nonionic surfactant; (iv) a volatile solvent, (v) water; (vi) at least one vasoconstrictor; (vii) a dimethicone/vinyldimethicone cross-polymer; and (viii) a silicone gum blend. According to still further embodiments, the pharmaceutical liquid adhesive composition comprises: (i) a silicone resin film forming agent comprising siloxysilicate, silsesquioxane, or a derivative or a combination thereof; (ii) anionic surfactant; (iii) nonionic surfactant; (iv) a volatile solvent, (v) water; (vi) at least one vasoconstrictor; (vii) a dimethicone/vinyldimethicone cross-polymer; (viii) a silicone gum blend; and (ix) a silicone acrylate.

According to further embodiments, the pharmaceutical liquid adhesive composition comprises: (i) a silicone resin film forming agent comprising siloxysilicate, silsesquioxane, or a derivative or a combination thereof; (ii) nonionic surfactant; (iii) a volatile solvent, (iv) water; (v) at least one vasoconstrictor; (vi) a dimethicone/vinyldimethicone cross-polymer; and (vii) a silicone gum blend. According to still further embodiments, the pharmaceutical liquid adhesive composition comprises: (i) a silicone resin film forming agent comprising siloxysilicate, silsesquioxane, or a derivative or a combination thereof; (ii) nonionic surfactant; (iii) a volatile solvent, (iv) water; (v) at least one vasoconstrictor; (vi) a dimethicone/vinyldimethicone cross-polymer and (vii) a silicone gum blend.

According to yet further embodiments, the pharmaceutical liquid adhesive composition comprises: (i) trimethylsiloxysilicate; (ii) sodium alkyl sulfate; (iii) a volatile solvent, selected from the group consisting of polydimethylsiloxane, aliphatic hydrocarbon and a combination thereof; (iv) water; (v) at least one vasoconstrictor; (vi) bis-vinyldimethicone, vinyldimethicone and hydrogen dimethicone; and (vii) dimethiconol and silicone oil blend. According to still further embodiments, the pharmaceutical liquid adhesive composition comprises: (i) trimethylsiloxysilicate; (ii) sodium alkyl sulfate; (iii) a volatile solvent, selected from the group consisting of polydimethylsiloxane, aliphatic hydrocarbon and a combination thereof; (iv) water; (v) at least one vasoconstrictor; (vi) bis-vinyldimethicone, vinyldimethicone and hydrogen dimethicone; (vii) dimethiconol and silicone oil blend; and (iv) a silicone acrylate.

According to yet further embodiments, the pharmaceutical liquid adhesive composition comprises: (i) trimethylsiloxysilicate; (ii) sodium alkyl sulfate; (iii) alkyl- and alkoxy-dimethicone copolyol; (iv) a volatile solvent, selected from the group consisting of polydimethylsiloxane, aliphatic hydrocarbon and a combination thereof; (v) water; (vi) at least one vasoconstrictor; (vii) bis-vinyldimethicone, vinyldimethicone and hydrogen dimethicone; and (viii) dimethiconol and silicone oil blend. According to still further embodiments, the pharmaceutical liquid adhesive composition comprises: (i) trimethylsiloxysilicate; (ii) sodium alkyl sulfate; (iii) alkyl- and alkoxy-dimethicone copolyol; (iv) a volatile solvent, selected from the group consisting of polydimethylsiloxane, aliphatic hydrocarbon and a combination thereof; (v) water; (vi) at least one vasoconstrictor; (vii) bis-vinyldimethicone, vinyldimethicone and hydrogen dimethicone; (viii) dimethiconol and silicone oil blend; and (ix) silicone acrylates.

According to yet further embodiments, the pharmaceutical liquid adhesive composition comprises: (i) trimethylsiloxysilicate; (ii) polysorbate; (iii) alkyl- and alkoxy-dimethicone copolyol; (iv) a volatile solvent, selected from the group consisting of polydimethylsiloxane, aliphatic hydrocarbon and a combination thereof; (v) water; (vi) at least one vasoconstrictor; (vii) bis-vinyldimethicone, vinyldimethicone and hydrogen dimethicone; and (viii) dimethiconol and silicone oil blend. According to still further embodiments, the pharmaceutical liquid adhesive composition comprises: (i) trimethylsiloxysilicate; (ii) polysorbate; (iii) alkyl- and alkoxy-dimethicone copolyol; (iv) a volatile solvent, selected from the group consisting of polydimethylsiloxane, aliphatic hydrocarbon and a combination thereof; (v) water; (vi) at least one vasoconstrictor; (vii) bis-vinyldimethicone, vinyldimethicone and hydrogen dimethicone; (viii) dimethiconol and silicone oil blend; and (ix) a silicone acrylate.

According to yet further embodiments, the pharmaceutical liquid adhesive composition comprises: (i) trimethylsiloxysilicate; (ii) sodium lauryl sulfate; (iii) a volatile solvent, selected from the group consisting of hexamethyldisiloxane, isooctane and a combination thereof; (iv) water; (v) at least one vasoconstrictor, selected from the group consisting of an anesthetic agent, a vasoconstrictor, an antipruritic agent, an immunomodulator, a cytotoxin, an anti-inflammatory agent, a muscle relaxant, and a combination thereof; (vi) bis-vinyldimethicone, vinyldimethicone and hydrogen dimethicone; and (vii) cyclopentasiloxane and dimethiconol. According to still further embodiments, the pharmaceutical liquid adhesive composition comprises: (i) trimethylsiloxysilicate; (ii) sodium lauryl sulfate; (iii) a volatile solvent, selected from the group consisting of hexamethyldisiloxane, isooctane and a combination thereof; (iv) water; (v) at least one vasoconstrictor, selected from the group consisting of vasoconstrictors and combinations thereof; (vi) bis-vinyldimethicone, vinyldimethicone and hydrogen dimethicone; (vii) cyclopentasiloxane and dimethiconol; and (iv) a silicone acrylate.

According to yet further embodiments, the pharmaceutical liquid adhesive composition comprises: (i) trimethylsiloxysilicate; (ii) sodium lauryl sulfate; (iii) Cetyl PEG/PPG-10/1 Dimethicone; (iv) a volatile solvent, selected from the group consisting of hexamethyldisiloxane, isooctane and a combination thereof; (v) water; (vi) at least one vasoconstrictor selected from the group comprising vasoconstrictors or combinations thereof; (vii) bis-vinyldimethicone, vinyldimethicone and hydrogen dimethicone; and (viii) cyclopentasiloxane and dimethiconol.

According to still further embodiments, the pharmaceutical liquid adhesive composition comprises: (i) trimethylsiloxysilicate; (ii) sodium lauryl sulfate; (iii) Cetyl PEG/PPG-10/1 Dimethicone; (iv) a volatile solvent, selected from the group consisting of hexamethyldisiloxane, isooctane and a combination thereof; (v) water; (vi) at least one vasoconstrictor selected from the group consisting of an vasoconstrictor, an (vii) bis-vinyldimethicone, vinyldimethicone and hydrogen dimethicone; (viii) cyclopentasiloxane and dimethiconol; and (ix) a silicone acrylate.

According to yet further embodiments, the pharmaceutical liquid adhesive composition comprises: (i) trimethylsiloxysilicate; (ii) Tween 80; (iii) Cetyl PEG/PPG-10/1 Dimethicone; (iv) a volatile solvent, selected from the group consisting of hexamethyldisiloxane, isooctane and a combination thereof; (v) water; (vi) at least one vasoconstrictor selected from the group consisting, a vasoconstrictor; (vii) bis-vinyldimethicone, vinyldimethicone and hydrogen dimethicone; and (viii) cyclopentasiloxane and dimethiconol. According to still further embodiments, the pharmaceutical liquid adhesive composition comprises: (i) trimethylsiloxysilicate; (ii) Tween 80; (iii) Cetyl PEG/PPG-10/1 Dimethicone; a silicone acrylate (iv) a volatile solvent, selected from the group consisting of hexamethyldisiloxane, isooctane and a combination thereof; (v) water; (vi) at least one vasoconstrictor selected from the group consisting of, a vasoconstrictor, (vii) bis-vinyldimethicone, vinyldimethicone and hydrogen dimethicone; (viii) cyclopentasiloxane and dimethiconol; and (ix) a silicone acrylate.

According to some embodiments, the pharmaceutical liquid adhesive composition comprises: (i) about 10-40% w/w of a silicone resin film forming agent comprising a silicone acrylate, siloxysilicate, silsesquioxane, or a derivative or a combination thereof; (ii) about 0.5-7% w/w of a surfactant; (iii) about 30-80% w/w of a volatile solvent; (iv) about 20-40% w/w of water; and (v) about 0.005-25% w/w of at least one vasoconstrictor. According to further embodiments, the pharmaceutical liquid adhesive composition comprises: (i) about 10-40% w/w of a silicone resin film forming agent comprising siloxysilicate, silsesquioxane, or a derivative or a combination thereof; (ii) about 0.5-2.5% w/w of an anionic surfactant; (iii) about 30-80% w/w of a volatile solvent; (iv) about 20-40% w/w of water; and (v) about 0.005-25% w/w of at least one vasoconstrictor. According to additional embodiments, the pharmaceutical liquid adhesive composition comprises: (i) about 10-40% w/w of a silicone resin film forming agent comprising siloxysilicate, silsesquioxane, or a derivative or a combination thereof; (ii) about 0.5-2.5% w/w of an anionic surfactant; (iii) about 30-80% w/w of a volatile solvent; (iv) about 20-40% w/w of water; (v) about 0.005-25% w/w of at least one vasoconstrictor; and (vi) about 0.05 -5% w/w of a silicone acrylate.

According to additional embodiments, the pharmaceutical liquid adhesive composition comprises: (i) about 10-40% w/w of a silicone resin film forming agent comprising a silicone acrylate, siloxysilicate, silsesquioxane, or a derivative or a combination thereof; (ii) about 0.5-2.5% w/w of an anionic surfactant; (iii) about 2-7% w/w of a nonionic surfactant; (iv) about 30-50% w/w of a volatile solvent; (v) about 25-40% w/w of water; and (vi) about 0.005-25% w/w of at least one vasoconstrictor. According to further embodiments, the pharmaceutical liquid adhesive composition comprises: (i) about 10-40% w/w of a silicone resin film forming agent comprising siloxysilicate, silsesquioxane, a silicone acrylate or a derivative or a combination thereof; (ii) about 0.5-2.5% w/w of an anionic surfactant; (iii) about 2-7% w/w of a nonionic surfactant; (iv) about 30-80% w/w of a volatile solvent; (v) about 20-40% w/w of water; (vi) about 0.005-25% w/w of at least one vasoconstrictor and (vii) silicon acrylate.

According to additional embodiments, the pharmaceutical liquid adhesive composition comprises: (i) about 10-40% w/w of a silicone resin film forming agent comprising a silicone acrylate, siloxysilicate, silsesquioxane, or a derivative or a combination thereof; (ii) about 0.5-7% w/w of a nonionic surfactant; (iii) about 30-80% w/w of a volatile solvent; (iv) about 20-40% w/w of water; and (v) about 0.005-25% w/w of at least one vasoconstrictor. According to further embodiments, the pharmaceutical liquid adhesive composition comprises: (i) about 10-40% w/w of a silicone resin film forming agent comprising siloxysilicate, silsesquioxane, or a derivative or a combination thereof; (ii) about 0.5-7% w/w of a nonionic surfactant; (iii) about 30-80% w/w of a volatile solvent; (iv) about 20-40% w/w of water; (v) a silicone acrylate and (vi) about 0.005-25% w/w of at least one vasoconstrictor.

According to additional embodiments, the pharmaceutical liquid adhesive composition comprises: (i) about 10-40% w/w of a silicone acrylate or trimethylsiloxysilicate; (ii) about 0.5-2.5% w/w of sodium alkyl sulfate; (iii) about 30-80% w/w of a volatile solvent, selected from the group consisting of polydimethylsiloxane, aliphatic hydrocarbon and a combination thereof; (iv) about 20-40% w/w of water; (v) about 0.005-25% w/w of at least one vasoconstrictor, selected from the group consisting of an, a vasoconstrictor, (vi) about 5-15% w/w bis-vinyldimethicone, vinyldimethicone and hydrogen dimethicone; and (vii) about 0.5-2.5% w/w dimethiconol and silicone oil blend, a silicone acrylate.

According to additional embodiments, the pharmaceutical liquid adhesive composition comprises: (i) about 10-40% w/w of trimethylsiloxysilicate; (ii) about 0.5-2.5% w/w of sodium alkyl sulfate; (iii) about 30-80% w/w of a volatile solvent, selected from the group consisting of polydimethylsiloxane, aliphatic hydrocarbon and a combination thereof; (iv) about 20-40% w/w of water; (v) about 0.005-25% w/w of at least one vasoconstrictor, selected from the group consisting of, a vasoconstrictor; (vi) about 5-15% w/w bis-vinyldimethicone, vinyldimethicone and hydrogen dimethicone; (vii) about 0.5-2.5% w/w dimethiconol and silicone oil blend; and (viii) about 0.05-5% w/w of a silicone acrylate.

According to additional embodiments, the pharmaceutical liquid adhesive composition comprises: (i) about 10-40% w/w of trimethylsiloxysilicate; (ii) about 0.5-2.5% w/w of sodium alkyl sulfate; (iii) about 2-7% w/w of alkyl- and alkoxy-dimethicone copolyol; (iv) about 30-80% w/w of a volatile solvent, selected from the group consisting of polydimethylsiloxane, aliphatic hydrocarbon and a combination thereof; (v) about 20-40% w/w of water; (vi) about 0.005-25% w/w of at least one vasoconstrictor, selected from the group consisting of a vasoconstrictor, (vii) about 5-15% w/w bis-vinyldimethicone, vinyldimethicone and hydrogen dimethicone; and (viii) about 0.5-2.5% w/w dimethiconol and a silicone acrylate.

According to additional embodiments, the pharmaceutical liquid adhesive composition comprises: (i) about 10-40% w/w of trimethylsiloxysilicate; (ii) about 0.5-2.5% w/w of sodium alkyl sulfate; (iii) about 2-7% w/w of alkyl- and alkoxy-dimethicone copolyol; (iv) about 30-80% w/w of a volatile solvent, selected from the group consisting of polydimethylsiloxane, aliphatic hydrocarbon and a combination thereof; (v) about 20-40% w/w of water; (vi) about 0.005-25% w/w of at least one vasoconstrictor, selected from the group consisting of vasoconstrictors or combinations thereof, (vii) about 5-15% w/w bis-vinyldimethicone, vinyldimethicone and hydrogen dimethicone; (viii) about 0.5-2.5% w/w dimethiconol and silicone oil blend and (viii) about 0.05-5% of a silicone acrylate.

According to additional embodiments, the pharmaceutical liquid adhesive composition comprises: (i) about 10-40% w/w of trimethylsiloxysilicate; (ii) about 0.5-2.5% w/w of polysorbate; (iii) about 2-7% w/w of alkyl- and alkoxy-dimethicone copolyol; (iv) about 30-80% w/w of a volatile solvent, selected from the group consisting of polydimethylsiloxane, aliphatic hydrocarbon and a combination thereof; (v) about 20-40% w/w of water; (vi) about 0.005-25% w/w of at least one vasoconstrictor, selected from the group consisting of, a vasoconstrictor, (viii) about 0.5-2.5% w/w dimethiconol and a silicone acrylate.

According to additional embodiments, the pharmaceutical liquid adhesive composition comprises: (i) about 10-40% w/w of trimethylsiloxysilicate; (ii) about 0.5-2.5% w/w of polysorbate; (iii) about 2-7% w/w of alkyl- and alkoxy-dimethicone copolyol; (iv) about 30-80% w/w of a volatile solvent, selected from the group consisting of polydimethylsiloxane, aliphatic hydrocarbon and a combination thereof; (v) about 20-40% w/w of water; (vi) about 0.005-25% w/w of at least one vasoconstrictor, selected from the group consisting of vasoconstrictors or mixtures thereof (vii) about 5-15% w/w bis-vinyldimethicone, vinyldimethicone and hydrogen dimethicone; (viii) about 0.5-2.5% w/w dimethiconol and silicone oil blend and (viii) about 1-15% of a silicone acrylate.

According to certain embodiment, the pharmaceutical liquid adhesive composition comprises: (i) about 15% w/w trimethylsiloxysilicate; (ii) about 1.5% w/w sodium lauryl sulfate; (iii) about 22% w/w hexamethyldisiloxane and 21% w/w isooctane; (iv) about 25% w/w water; (v) about 0.05% w/w phenylephrine; (vi) about 5% w/w bis-vinyldimethicone and 5% w/w vinyldimethicone and hydrogen dimethicone; and (vii) about 1% w/w cyclopentasiloxane and dimethiconol.

According to another embodiment, the pharmaceutical liquid adhesive composition comprises: (i) about 15% w/w trimethylsiloxysilicate; (ii) about 1.5% w/w sodium lauryl sulfate; (iii) about 22% w/w hexamethyldisiloxane and 21% w/w isooctane; (iv) about 25% w/w water;

(v) about 0.05% w/w phenylephrine; (vi) about 5% w/w bis-vinyldimethicone and 5% w/w vinyldimethicone and hydrogen dimethicone; (vii) about 1% w/w cyclopentasiloxane and dimethiconol; and (viii) about 5% w/w of a silicone acrylate.

According to still another embodiment, the pharmaceutical liquid adhesive composition comprises: (i) about 15% w/w trimethylsiloxysilicate; (ii) about 1.5% w/w sodium lauryl sulfate;

(iii) about 4% w/w Cetyl PEG/PPG-10/1 Dimethicone; (iv) about 22% w/w hexamethyldisiloxane and 21% w/w isooctane; (v) about 25% w/w water; (vi) about 0.05% w/w phenylephrine; (vii) about 5% w/w bis-vinyldimethicone and 5% w/w vinyldimethicone and hydrogen dimethicone; and (viii) about 1% w/w cyclopentasiloxane and dimethiconol.

According to yet another embodiment, the pharmaceutical liquid adhesive composition comprises: (i) about 15% w/w trimethylsiloxysilicate; (ii) about 1.5% w/w sodium lauryl sulfate; (iii) about 4% w/w Cetyl PEG/PPG-10/1 Dimethicone; (iv) about 18% w/w hexamethyldisiloxane and 19% w/w isooctane; (v) about 30% w/w water; (vi) about 0.05% w/w phenylephrine; (vii) about 5% w/w bis-vinyldimethicone and 5% w/w vinyldimethicone and hydrogen dimethicone; (viii) about 1% w/w cyclopentasiloxane and dimethiconol; and (ix) about 0.5% w/w hydroxypropyl methyl cellulose.

According to still another embodiment, the pharmaceutical liquid adhesive composition comprises: (i) about 15% w/w trimethylsiloxysilicate; (ii) about 1.5% w/w Tween 80; (iii) about 4% w/w Cetyl PEG/PPG-10/1 Dimethicone; (iv) about 22% w/w hexamethyldisiloxane and 21% w/w isooctane; (v) about 25% w/w water; (vi) about 1% w/w oxymethazoline (vii) about 0.05% w/w phenylephrine; (viii) about 5% w/w bis-vinyldimethicone and 5% w/w vinyldimethicone and hydrogen dimethicone; and (ix) about 1% w/w cyclopentasiloxane and dimethiconol.

According to yet another embodiment, the pharmaceutical liquid adhesive emulsion comprises: (i) about 15% w/w trimethylsiloxysilicate; (ii) about 1.5% w/w Tween 80; (iii) about 4% w/w Cetyl PEG/PPG-10/1 Dimethicone; (iv) about 18% w/w hexamethyldisiloxane and 19% w/w isooctane; (v) about 30% w/w water; (vi) about 0.05% w/w phenylephrine; (vii) about 5% w/w bis-vinyldimethicone and 5% w/w vinyldimethicone and hydrogen dimethicone; (viii) about 1% w/w cyclopentasiloxane and dimethiconol; and (ix) about 0.5% w/w of a silicone acrylate

According to an embodiment, there is provided a topical composition for the treatment and prevention of all types of nosebleed (Epistaxis) comprising
(i) from 15.0% to 30% of at least one silicon resin film forming agent selected from the group consisting of siloxysilicates, silicone acrylates and combinations thereof
(ii) from 30% to 75% of at least one volatile solvent selected from the group consisting of non-polar volatile siloxanes, volatile aliphatic hydrocarbons, volatile hydrofluoroalkanes and combinations thereof;
(iii) from 0.05% (w/w) to 20% of at least one vasoconstrictor active agent selected from the group consisting of phenylephrine, epinephrine, tetrahydrozoline, an amphetamine, an antihistamine, methylphenidate, mephedrone, oxymetazoline, pseudoephedrine, psilocybin, ephedrine, their salts and combinations thereof.

According to another embodiment, there is provided a composition as detailed above, wherein further comprising from 0.05% to 20% of at least one more active agent, wherein the at least one more active agent is an antifibrinolytic active agent selected from tranexamic acid, aprotinin, ε-aminocaproic acid, aminomethylbenzoic acid or their salts and combinations thereof The compositions may optionally further comprise from 15% (w/w) to 40% (w/w) of water or a buffer and from 1% (w/w) to 5% of at least one surfactant selected from the group consisting of sodium lauryl sulfate, alkyl- and alkoxy-dimethicone copolyol, polysorbate and a combination thereof. Such compositions may be administered in the form of a gel or nasal swab/wipe.

The at least one siloxysilicate may be trimethylsiloxysilicate.

The above at least one vasoconstrictor may be phenylephrine, its hydrochloride or combinations thereof.

The at least one antifibrinolytic may be tranexamic acid, its salt or combinations thereof.

In an embodiment, there is provided a composition comprising from 0.05% to 2% of phenylephrine or its hydrochloride and from 3% to 10% tranexamic acid or its salt.

In another embodiment, there is provided a composition comprising about 0.25% w/w of phenylephrine or its hydrochloride and about 5% w/w tranexamic acid or its salt.

The non-polar volatile siloxanes are selected from the group consisting of hexamethyldisiloxane, heptamethyloctyltrisiloxane, octamethylcyclotetrasiloxane, octamethyltrisiloxane, decamethylcyclopentasiloxane, decamethyltetrasiloxane, dodecamethylpentasiloxane, dodecamethylcyclohexasiloxane, and combinations thereof.

The volatile aliphatic hydrocarbons are selected from the group consisting of pentane, isooctane, isododecane, isohexadecane and combinations thereof.

The volatile hydrofluoroalkanes are selected from the group consisting of 1,1,1,2-tetrafluoroethane (HFA 134a), 1,1,1,2,3,3,3-heptafluoro-n-propane (HFA 227) and combinations thereof.

The at least one surfactant is a siliconic surfactant and optionally an additional surfactant is anionic.

The optional additional anionic surfactant is selected from the group consisting of sodium alkyl sulfate, sodium alkyl sulfonate, sodium alkyl aryl sulfonate, sodium stearate, dioctyl sodium sulfosuccinate, sodium cholate, and any combination thereof.

The sodium alkyl sulfate may be sodium lauryl sulfate. The at least one surfactant may be a nonionic surfactant.

The nonionic surfactant is selected from the group consisting of organosilicon surfactants, nonionic organic surfactants, and combinations thereof.

The organosilicon surfactant is selected from the group comprising alkyl- and alkoxy-dimethicone copolyol.

A alkyl- and alkoxy-dimethicone copolyol is cetyl dimethicone copolyol.

A cetyl dimethicone copolyol is Cetyl PEG/PPG-10/1 Dimethicone.

The nonionic organic surfactant is selected from the group consisting of polysorbate, glyceryl stearate, polyoxyethylene (POE) fatty acid ester, poly(oxyethylene) alkylyl ether, polyethoxylene castor oil derivative, PEG-6 octanoic/decanoic glycerides, polyoxyethylene glycerol trioleate, decaglycerol mono/dioleate, and any combination thereof.

The polysorbate is selected from the group consisting of polyoxyethylene sorbitan monolaurate (Tween 20), polyoxyethylene sorbitan monopalmitate (Tween 40), polyoxyethylene sorbitan monostearate (Tween 60) and polyoxyethylene sorbitan monooleate (Tween 80).

The at least one surfactant is selected from the group consisting of a cationic surfactant, an amphoteric surfactant, and a combination thereof.

In an embodiment, there is provided a composition comprising
(i) from 10.0% (w/w) to 30.0% (w/w) of a silicone acrylate;
(ii)from 1.0% (w/w) to 5.0% (w/w) of at least one surfactant selected from the group consisting of siliconic surfactants, anionic surfactants, nonionic surfactants, and combinations thereof;
(iii) from 30.0% (w/w) to 75.0% (w/w) of a volatile solvent selected from the group consisting of a polydimethylsiloxane, an aliphatic hydrocarbon, and combinations thereof;
(iv) from 15% (w/w) to 40% (w/w) of water; and
(v) from 0.005% (w/w) to about 25.0% (w/w) of a vasoconstrictor selected from the group consisting of phenylephrine, phenylephrine, epinephrine, epinephrine, tetrahydrozoline, an amphetamine, an antihistamine, methylphenidate, mephedrone, oxymetazoline, pseudoephedrine, psilocybin, ephedrine their salts and combinations thereof.

In another embodiment, there is provided a composition comprising
(i) about 10-40% w/w of trimethylsiloxysilicate;
(ii) about 0.5-7% w/w of a surfactant selected from the group consisting of sodium lauryl sulfate, alkyl- and alkoxy-dimethicone copolyol, polysorbate and a combination thereof;
(iii) about 30-80% w/w of a volatile solvent selected from the group consisting of hexamethyldisiloxane, isooctane and combinations thereof;
(iv) from 20% (w/w) to 40% (w/w) of water; and
(v) from 0.005% w/w to 25% w/w of a vasoconstrictor selected from the group consisting of phenylephrine, phenylephrine hydrochloride, epinephrine, epinephrine hydrochloride, tetrahydrozoline hydrochloride, an amphetamine, an antihistamine, methylphenidate, mephedrone, oxymetazoline, pseudoephedrine, psilocybin, ephedrine sulfate, and combinations thereof.

In another embodiment, there is provided a composition comprising
(i) from 15% w/w to 20% w/w trimethylsiloxysilicate;
(ii) from 1.5% w/w to 3.0% w/w sodium lauryl sulfate;
(iii) from 22% w/w to 30% w/w hexamethyldisiloxane and from 20-25% w/w isooctane;
(iv) from 25% w/w to 30% w/w water;
(v) from 10% w/w to 15% of a silicone acrylate
(vi) from 0.05% w/w to 0.25% w/w phenylephrine hydrochloride.

In yet another embodiment, there is provided a composition comprising
(i) from 15% w/w to 20% w/w trimethylsiloxysilicate;
(ii) from 1.5% w/w to 3.0% w/w sodium lauryl sulfate;
(iii) from 22% w/w to 30% w/w hexamethyldisiloxane and from 20-25% w/w isooctane;
(iv) from 25% w/w to 30% w/w water;
(v) from 10% w/w to 15% of a silicone acrylate
(vi) 0.05% w/w to 0.25% w/w of phenylephrine hydrochloride.
(vii) from 3% w/w to 10% w/w of tranexamic acid

The composition of the instant disclosure may be administered to a subject in need thereof in the form of nasal swab, a single use wipe, a gel, a nasal spray, a foam, a towelette, a syringe, a dropper, a spray dispenser, a compressible bottle or tube, a spatula, a suppository insertion tube, an extrusion tube, and an inflatable member.

In an embodiment, there is provided a method of treatment or prevention of any type of nose bleeding, including an Epistaxis disorder, the method comprising the step of topically applying to the mucosal surface of the affected nostril of a subject in need of such treatment a therapeutically effective amount of the composition of the instant disclosure.

There is provided a kit comprising a pharmaceutical liquid adhesive composition and a container-applicator device suitable for storage and application of the composition to the nose, into the affected nostril.

The container-applicator device the kit comprises at least one of a single use wipe, a towelette, a syringe, a dropper, a spray dispenser, a compressible bottle or tube, a spatula, a suppository insertion tube, an extrusion tube, and an inflatable member.

In an embodiment, there is provided a pharmaceutical liquid adhesive composition of the instant disclosure for use in preventing or treating any type of nose bleeding, including an Epistaxis disorder.

### Mode of Administration, Containers and Applicators

The compositions for use in the present invention are generally stored in a container-applicator device for use in a single dose application or for use in repeated applications to the nose. Single dose applicators include those having breakable or removable seals that prevent moisture, including atmospheric moisture, from contacting the formulation.

The compositions may be administered to an Epistaxis affected subject in need thereof in the form of gel, nasal swab/wipe or nasal spray/aerosol.

In an embodiment, the liquid adhesive is comprised in the pre-packaged nasal swab/towelette/wipe. The nasal swab/wipe substrate is uniformly impregnated with the liquid adhesive composition. According to some embodiments, the liquid adhesive composition is in a liquid form, when applied to the wipe. According to other embodiments, the liquid adhesive composition is in a gel form, when applied to said wipe. The wipe provides the user with a single dose of sterile medication. The liquid adhesive is transferred to the nostril upon contacting the nasal swab/wipe with the nostril surface.

Each wipe is generally packaged as a single-use sealed unit. The wipe is formed of woven or non-woven fabric, cloth or tissue substrate and the impregnated wipe is sealed into an enveloping sachet or pocket. The sachet or pocket is formed by sandwiching a folded and impregnated wipe between two sheets of an aluminum foil/polyethylene film laminate. The sheets of laminate may comprise folded over portions of a single sheet of such material.

A container-applicator may further comprise two parts: (1) a storage area or reservoir which holds the composition and protects it from air, water and contaminants; and (2) the applicator which generally comprises a specially shaped tip designed to aid in application of the composition to the nose. In particular embodiments, the applicator is an element integral to the container, for example, an elongated insertion tube extending from a reservoir. Alternately, the storage area and the applicator may be separate components, such as a tube reservoir and a separately supplied dropper. In yet other embodiments, the container and the applicator may be supplied as separate elements which are connected during use, for example via compatible male and female connectors respectively provided on the container and the applicator or vice versa.

For repeated and intermittent usage, minimal exposure to atmospheric moisture is required. This can be achieved by devices having very narrow applicator outlets and low initial dead space. One applicator for such repeated intermittent use dispenses the adhesive in a controlled drop wise manner, as described for example in U.S. Pat. No. 4,958,748, which is hereby incorporated by reference in its entirety.

Still another container-applicator device comprises a brush or solid paddle applicator wherein the liquid adhesive is “painted” onto the nostril surface requiring treatment.

The container-applicator device for repeated and intermittent usage may comprise a container suitable for non-sterile storage of the composition, and an applicator suitable for metered dispensement of the composition after opening of the applicator. In particular embodiments, the applicator is characterized as having a resealable opening of no more than about 0.05 square inch (0.323 square centimeters) so as to permit the metered dispensement of the adhesive from the applicator and which is capable of multiple administrations of the adhesive, and is further characterized as having resealing means such as a cap which either tightly mates with the applicator or which screws onto the applicator. The opening may be at the terminus of an elongated and tapered tube-like member suitable for insertion into the nose. The opening of the applicator is about 0.001 to about 0.01 square inch (about 0.00645 to about 0.0645 square centimeters).

In another embodiment, the walls of the container-applicator device are made of a pliable material, so that upon application of pressure onto the walls, the walls depress sufficiently to force the adhesive in the container into the applicator and through the opening. In another embodiment, the adhesive is released from the applicator by gravity feed methods. Such methods do not require application of pressure to the walls of the container.

The applicator is manufactured with its opening covered by a metal foil or other similar construction which closes this opening until the device is ready for use. The opening is then reinstated by use of a pin or similar device which punctures the covering.

Such devices for intermittent use enable multiple uses of the liquid adhesive at different points in time by the same individual.

In container-applicator devices suitable for repeated intermittent uses, the liquid adhesive is stored at ambient conditions and is selected to be bacteriostatic. See, for example, U.S. Pat. No. 3,527,224, which is hereby incorporated by reference in its entirety. When the selected adhesive is bacteriostatic, prolonged storage at ambient conditions can be achieved without regard to the sterility of the formulation because there is no adverse buildup of bacteria during storage.

The reservoir of the container-applicator device is both air-tight and water-tight, and keeps the media within free from contaminants. The reservoir may contain a desiccant material to keep the media free of water. Reservoirs may be of any shape, for example, the shape is configured to allow a smooth internal flow of media, such as a cylindrical or conical shape. The size of the reservoir may vary within a wide range, but is slightly larger than the volume of composition which will be placed inside the reservoir to minimize the amount of gas within the reservoir. The reservoir may be made from any of a variety of medical grade materials, such as plastics, excluding glass. Vasoconstrictors of the liquid adhesive suffer from caking when stored in glass reservoir. The reservoir may be either rigid, collapsible, or compressible. Use of a compressible or collapsible reservoir allows the user to have greater control over the rate at which the composition is expressed, as exertion of pressure on a compressible or collapsible reservoir would place a force on the on the composition causing it to flow at a faster rate than it would in the absence of such pressure. The compressible or collapsible reservoir design can allow for the liquid adhesive composition in the form of gel, for which the force of gravity may not be strong enough to cause a flow through an applicator sufficient to treat Epistaxis. Collapsible reservoirs which retain their collapsed shape have the additional advantage of reducing the amount of air which enters the reservoir following use. This advantage of collapsible containers is of greater importance in multiple-use (reusable) devices, wherein media is kept relatively free of potential contaminants between uses.

Applicator tips can be of any of a number of shapes, sizes, and configurations. They are fairly rigid and may be made out of any material which is compatible with the media formulation, e.g., but not limited to, plastic, excluding glass. The choice of a proper applicator tip for a given application will depend on factors such as the viscosity of the composition, the desired application rate of the composition, the nature of the Epistaxis disorder, and its severity.

The container-applicators of the present invention may be either single-use or multiple-use devices. A container or reservoir containing enough liquid adhesive composition for multiple applications may be configured to accommodate replaceable tips. In such an embodiment, at the place whereupon the replaceable tips connect with the reservoir, the reservoir would have a means such as a valve, septum or sealing gasket which allows the reservoir to be sealed in the absence of an applicator tip. Placing an applicator tip on the reservoir would cause the valve to open, allowing composition to flow out from the reservoir. In this manner, one reservoir containing enough composition for several applications could be used over a period of hours, days or weeks. This embodiment would also allow the user to use one reservoir with applicator tips of varying shapes and sizes chosen to best accommodate the Epistaxis disorder during the healing process.

In an embodiment, there are provided nasal sprays/aerosols for the treatment and prevention of Epistaxis (see Examples 8-11).

The nasal sprays/aerosols of this disclosure are suspensions of the pharmaceutical active agents selected from the group consisting of vasoconstrictors, antifibrinolytics and optionally anti-inflammatory agents in at least one HFA (hydrofluoroalkane) containing a film forming agent. As the aerosol is intended for local topical use in the nostril and not for inhalation to the lungs, the nozzle is best designed for delivering a wide plume of aerosol, to be deposited laterally on the nostril walls.

The nasal sprays of this disclosure may be metered dose nasal sprays aerosols formulated with HFAs, aqueous nasal sprays or dry powder nasal sprays.

The compositions in the nasal sprays may be delivered in metered doses, also named actuations or “puffs’. A number of actuations (puffs) per day from a metered dose aerosol may be needed for the treatment and prevention of Epistaxis, according to doctor's instructions.

After evaporation of the HFA(s), the compositions leave on the nostril's surface a flexible and durable film which contains therapeutically effective doses of the active pharmaceutical ingredient(s). These active ingredients are delivered slowly over a period of time, affording an extended release effect and protection against nosebleeds. The film obtained affords in addition to the therapeutic effect of the active ingredients, also a physical occlusive effect.

The HFAs are selected from the group of FDA-approved hydrofluoroalkanes, including 1,1,1,2-tetrafluoroethane (HFA 134a), 1,1,1,2,3,3,3-heptafluoro-n-propane (HFA 227) and combinations thereof.

In some embodiments, the composition of the present invention is a HFA nasal spray/aerosol with a metering valve comprising:

(i) from 0.025% w/w to 2% w/w micronised phenylephrine hydrochloride;

(ii) from 3% w/w to 10% w/w micronised tranexamic acid;

(iii) from 15% w/w to 30% w/w trimethylsiloxysilicate powder; and

(iv) 50% w/w to 75% w/w 1,1,1,2-tetrafluoroethane.

### Uses

The present invention provides compositions which are useful for effectively treating a variety of Epistaxis disorders caused by injury to the nose, hemophilia, upper respiratory infection, hypertension, antiplatelet medication, foreign body, insufflated drugs, barotrauma, nasal surgery, nasal sprays, allergic reactions or combinations thereof.

Epistaxis disorders are commonly encountered among the general population, but are often inadequately unaddressed, since many patients delay or fail to seek medical attention. Furthermore, many medications for such conditions fail to provide adequate relief and healing. In addition, many medications which are intended for treatment of conditions such as Epistaxis may be difficult to self-administer, and are unsatisfactory due to their uncomfortable sensation after application.

The present invention provides compositions which are useful for effectively treating a variety of Epistaxis disorders, wherein the compositions provide enhanced therapeutic efficacy and are associated with improved patient compliance, as compared to prior art compositions. The provided compositions may be useful for simultaneously treating a number of Epistaxis disorders.

The term “therapeutically effective amount” as used herein means an amount of the vasoconstrictor which is sufficient to provide a beneficial effect to the subject to which the vasoconstrictor is administered. More specifically, a therapeutically effective amount means an amount of the vasoconstrictor effective to alleviate or ameliorate the symptoms of an Epistaxis disorder of the subject being treated.

After an initial layer of liquid adhesive has been applied and the solvent has evaporated, providing an initial adhesive coating, a second layer may be applied over the initial film. Additional amounts of liquid adhesive can be applied as needed.

Sufficient liquid adhesive is employed to form a coating of less than about 0.5 mm thick and at least about 0.1 mm thick. Such coatings can be formed by applying, for example, about 0.02 ml of liquid adhesive per square centimeter of affected surface area. In some embodiments, the liquid adhesive can form a coating (e.g., a film) of between 0.1-0.5 mm thick. In some embodiments, the liquid adhesive can form a coating (e.g., a film) of between 0.1-0.3 mm thick. In some embodiments, the liquid adhesive can form a coating (e.g., a film) of between 0.3-0.5 mm thick. In some embodiments, the liquid adhesive can form a coating (e.g., a film) of between 0.2-0.4 mm thick.

In general, the particular length of time required for film formation will vary depending on factors such as the amount of adhesive applied, the temperature of the Epistaxis mucosal area, the moisture content of the mucosal nasal area, the surface area for adhesive application, and the like. However, in an embodiment, film formation is generally complete within about 10 to about 60 seconds. In an embodiment, film formation is completed within about 10-30 seconds. In an embodiment, film formation is completed within about 30-60 seconds. In an embodiment, film formation is completed within about 15-45 seconds. During this period, the person to whom application of the liquid adhesive has been made minimizes actions and body movements thus allowing the adhesive to form a coating.

The liquid adhesive compositions are configured to act at room temperature (20° C.). The films are conformable and comfortable and may be elastic and flexible. The films do not irritate the skin and mucous membrane during the application and in use after drying. The liquid adhesives are substantially painless and easily removable substantially without pain. The dried films formed from the liquid adhesive compositions are substantially non-water sensitive and waterproof. The dried films formed from the liquid adhesive compositions comprise finely-dispersed pharmaceutical ingredients, which can be gradually released to the adhesion area.

The compositions of the present invention are applicable to both human patients and to non-human mammalian subjects such as in veterinary use, for example for treatment of canine, feline, equine, bovine, porcine and primate species.

All patents, patent applications, and published references cited herein are hereby incorporated by reference in their entirety. It will be appreciated that several of the above-disclosed and other features and functions, or alternatives thereof, may be desirably combined into many other different systems or application. Various presently unforeseen or unanticipated alternatives, modifications, variations, or improvements therein may be subsequently made by those skilled in the art.

The following examples illustrate certain embodiments of the invention but are not meant to limit the scope of the claims in any way.

### EXAMPLES

The following examples further illustrate the invention as it may be carried out but, of course, should not be construed as in any way limiting its scope.

### Example 1

### Liquid Composition for the Preparation of Nasal Swabs/Swipes

25 g (25% w/w) trimethylsiloxysilicate powder is dissolved in 38.25 g (38.25% w/w) methylsiloxane at room temperature. 4 g (4% w/w) Cetyl PEG/PPG-10/1 Dimethicone is added to the trimethylsiloxysilicate solution. 00.25 g (0.25%) phenylephrine hydrochloride is dissolved in water. The pH of the aqueous solution is adjusted to 4.2-4.4 with acetate buffer (30% w/w). 1.5 g (1.5% w/w) Tween 80 is added to the aqueous solution. The trimethylsiloxysilicate solution is combined with the aqueous solution and mix by means of a homogenizer at room temperature. The obtained topical liquid solution is applied to a wipe substrate and is sealed to provide a sealed package of single-use wipe impregnated with the topical liquid composition. The composition is configured to be applied to the Epistaxis affected nostril using single use wipe(s).

### Example 2

### Liquid Composition with Pemulen TR-1 for the Preparation of Nasal Swabs/Wipes

This composition is prepared similarly to composition of Example 1, with added Pemulen TR-1:

25 g (25%) trimethylsiloxysilicate powder is dissolved in 38.25 g (38.25% w/w) methylsiloxane at room temperature. 4 g (4% w/w) Cetyl PEG/PPG-10/1 Dimethicone is added to the trimethylsiloxysilicate solution. 0.25 g (0.25% w/w) phenylephrine hydrochloride is dissolved in water. The pH of the aqueous solution is adjusted to 4.2-4.4 using acetate buffer (30% w/w). 1.5 g (1.5% w/w) Tween 80 is added to the aqueous solution. The trimethylsiloxysilicate solution is combined with the aqueous solution and mixed by means of a homogenizer at room temperature. 0.1 g (0.1% w/w) Pemulen TR-1 is added and mixed by means of a homogenizer at room temperature.

A topical liquid composition is obtained, with a viscosity ranging from 1-1.2 cP, close to the viscosity of water.

The obtained topical liquid composition is applied to a wipe substrate and sealed to provide a sealed package of single-use wipe impregnated with the topical liquid composition. The composition is configured to be applied to the Epistaxis affected nostril using single use nasal swabs(s)/wipe(s).

### Example 3

### Gel Composition

25 g (25% w/w) trimethylsiloxysilicate powder is dissolved in 47.25 g (47.25% w/w) methylsiloxane at RT. 4 g (4% w/w) Silicon Surfactant Cetyl PEG/PPG-10/1 Dimethicone is added to the solution of trimethylsiloxysilicate. 0.25% g (0.25% w/w) phenylephrine hydrochloride is dissolved in water. The pH of the aqueous solution is adjusted to 4.2-4.4 using an acetate buffer 25 g (25% w/w). 1.5 g (1.5% w/w) Tween 80 is added to the aqueous solution with slow mixing to avoid bubbling. 0.6 g (0.6% w/w) Hydroxyethylcellulose (Natrosol HHX) is dispersed in the aqueous phase under intensive mixing and heated up to 70 deg C. The mixing continues after the mixture is obtained and is further continued until the mixture cools to room temperature. The trimethylsiloxysilicate solution is combined with the aqueous solution and mixed in a homogenizer at room temperature. Upon dissolution of hydroxypropyl methylcellulose in the aqueous phase, a viscous gel with a viscosity ranging from 25000-45000 cP is formed.

### Example 4

2.5 g (2.5% w/w) of sodium alkyl sulfate; 50 g (50% w/w) of the volatile solvent polydimethylsiloxane, 30 g (30% w/w) of water; (vi) 0.25 g (0.25% w/w) of phenylephrine hydrochloride, 12 g (12% w/w) of silicone acrylate (FA 4001-Dow) 5.4 g (5.4% w/w) of silicone surfactant (ES 5612-Dow) is mixed together in low shear mixer at room temperature and stored in a closed container protected from light.

### Example 5

1 g (1% w/w) of sodium alkyl sulfate; 50 g (50% w/w) of the volatile solvent polydimethylsiloxane, 30 g (30% w/w) of water; (vi) 0.1 g (0.1% w/w) of oxymethazoline, 12 g (12% w/w) of silicone acrylate (FA 4001-Dow), 6.9 g (6.9% w/w) of silicone surfactant (ES 5612-Dow) is mixed together in low shear mixer at room temperature and stored in a closed container protected from light.

### Example 6

50 g of the volatile solvent polydimethylsiloxane, 32.5 g (32.5% w/w) of water; (vi) 0.1 g (0.1% w/w) of phenylephrine hydrochloride, 12 g (12% w/w) of silicone acrylate (FA 4002-Dow), 5.4 g (5.4% w/w) of silicone surfactant (ES 5612-Dow) is mixed together in low shear mixer at room temperature and stored in a closed container protected from light.

### Example 7

70 g (70% w/w) of the volatile solvent polydimethylsiloxane, 10 g (10% w/w) of water; (vi) 0.1 g (0.1% w/w) of phenylephrine hydrochloride, 12 g (12% w/w) of silicone acrylate (FA 4001-Dow), 5.4 g (5.4% w/w) of silicone surfactant (ES 5612-Dow) is mixed together in low shear mixer at room temperature and stored in a closed container protected from light.

### Example 8

### Metered Aerosol Epistaxis Compositions with Propellant 134a, Tranexamic Acid and Phenylephrine

Micronised tranexamic acid (5.0 g, 5% w/w), micronised phenylephrine (0.25 g. 0.25% w/w), tranexamic acid (5 g, 5% w/w), trimethylsiloxysilicate powder (25.0 g, 25% w/w) and 1,1,1,2-tetrafluoroethane (69.75 g, 69.75% w/w) is weighed into a pressure vessel and mixed with a high shear mixer for 20 minutes to obtain a suspension. Aliquots (20 g) of the suspension are filled into aluminum cans closed with a metering valve, filling under pressure through the valve using conventional filling equipment. The resulting inhalers contain 1 g tranexamic acid and 0.05 g phenylephrine and deliver 100 puffs of 10 mg tranexamic acid and 0.5 mg phenylephrine per actuation.

### Example 9

### Metered Aerosol Epistaxis Compositions with Propellant 134a, Tranexamic Acid and Phenylephrine Hydrochloride

Micronised phenylephrine hydrochloride (0.25 g, 0.25% w/w), tranexamic acid (5 g, 5% w/w) trimethylsiloxysilicate powder (25.0 g, 25% w/w) and 1,1,1,2-tetrafluoroethane (74.75 g, 74.75% w/w) is weighed into a pressure vessel and mixed with a high shear mixer for 20 minutes to obtain a suspension. Aliquots (20 g) of the suspension are filled into aluminum cans closed with a metering valve, filling under pressure through the valve using conventional filling equipment. The resulting inhalers contain 0.05 g phenylephrine and deliver 100 puffs of 0.5 mg phenylephrine hydrochloride per actuation.

### Example 10

### Metered Aerosol Epistaxis Compositions with Propellant 134a and Phenylephrine

Micronised phenylephrine (0.25 g. 0.25% w/w), trimethylsiloxysilicate powder (25.0 g, 25% w/w) and 1,1,1,2-tetrafluoroethane (74.75 g, 74.75% w/w) are weighed into a pressure vessel and mixed with a high shear mixer for 20 minutes to obtain a suspension. Aliquots (20 g) of the suspension are filled into aluminum cans closed with a metering valve, filling under pressure through the valve using conventional filling equipment. The resulting inhalers contain 0.05 g phenylephrine and deliver 100 puffs of 0.5 mg phenylephrine per actuation.

### Example

### Metered Aerosol Epistaxis Compositions with Propellant 134a and Phenylephrine Hydrochloride

Micronised phenylephrine hydrochloride (0.25 g, 0.25% w/w), trimethylsiloxysilicate powder (25.0 g, 25% w/w) and 1,1,1,2-tetrafluoroethane (74.75 g, 74.75% w/w) are weighed into a pressure vessel and mixed with a high shear mixer for 20 minutes to obtain a suspension. Aliquots (20 g) of the suspension are filled into aluminum cans closed with a metering valve, filling under pressure through the valve using conventional filling equipment. The resulting inhalers contain 0.05 g phenylephrine and deliver 100 puffs of 0.5 mg phenylephrine hydrochloride per actuation.

What is claimed is:

1. A topical composition, comprising:
(i) from 15.0% to 30% of at least one silicon resin film forming agent selected from the group consisting of siloxysilicates, silicone acrylates and combinations thereof;
(ii) from 30% to 75% of at least one volatile solvent selected from the group consisting of non-polar volatile siloxanes, volatile aliphatic hydrocarbons, volatile hydrofluoroalkanes and combinations thereof; and
(iii) from 0.05% (w/w) to 20% of at least one vasoconstrictor active agent selected from the group consisting of phenylephrine, epinephrine, tetrahydrozoline, an amphetamine, an antihistamine, methylphenidate, mephedrone, oxymetazoline, pseudoephedrine, psilocybin, ephedrine, their salts and combinations thereof.

2. The composition of claim 1 further comprising from 0.05% to 20% of at least one additional active agent,
wherein the at least one additional active agent is an antifibrinolytic active agent selected from the group consisting of tranexamic acid, aprotinin, ε-aminocaproic acid, aminomethylbenzoic acid, their salts and combinations thereof.

3. The composition of claim 1, wherein the siloxysilicate is trimethylsiloxysilicate.

4. The composition of claim 1, wherein the at least one vasoconstrictor is phenylephrine, its hydrochloride or combinations thereof.

5. The composition of claim 2, wherein the antifibrinolytic is tranexamic acid.

6. The composition of claim 2, further comprising from 0.05% to 2% of phenylephrine or its hydrochloride and from 3% to 10% tranexamic acid or its salt.

7. The composition of claim 2, further comprising 0.25% w/w of phenylephrine or its hydrochloride and about 5% w/w tranexamic acid or its salt.

8. The composition of claim 1, wherein the non-polar volatile siloxanes are selected from the group consisting of hexamethyldisiloxane, heptamethyloctyltrisiloxane, octamethylcyclotetrasiloxane, octamethyltrisiloxane, decamethylcyclopentasiloxane, decamethyltetrasiloxane, dodecamethylpentasiloxane, dodecamethylcyclohexasiloxane, and any combination thereof.

9. The composition of claim 1, wherein the volatile aliphatic hydrocarbons are selected from the group consisting of pentane, isooctane, isododecane, isohexadecane and combinations thereof.

10. The composition of claim 1, wherein the volatile hydrofluoroalkanes are selected from the group consisting of 1,1,1,2-tetrafluoroethane (HFA 134a), 1,1,1,2,3,3,3-heptafluoro-n-propane (HFA 227) and combinations thereof.

11. The compositions of claim 1, further comprising from 15% (w/w) to 40% (w/w) of water or a buffer.

12. A composition comprising:
(i) from 10.0% (w/w) to 30.0% (w/w) of a silicone acrylate;
(ii) from 1.0% (w/w) to 5.0% (w/w) of at least one surfactant selected from the group consisting of siliconic surfactants, anionic surfactants, nonionic surfactants, and combinations thereof;
(iii) from 30.0% (w/w) to 75.0% (w/w) of a volatile solvent selected from the group consisting of a polydimethylsiloxane, an aliphatic hydrocarbon, and combinations thereof;
(iv) from 15% (w/w) to 40% (w/w) of water; and
(v) from 0.005% (w/w) to about 25.0% (w/w) of a vasoconstrictor selected from the group consisting of phenylephrine, phenylephrine, epinephrine, epinephrine, tetrahydrozoline, an amphetamine, an antihistamine, methylphenidate, mephedrone, oxymetazoline, pseudoephedrine, psilocybin, ephedrine their salts and combinations thereof.

13. A composition comprising:
(i) from 10-40% w/w of trimethylsiloxysilicate;
(ii) from 0.5-7% w/w of a surfactant selected from the group consisting of sodium lauryl sulfate, alkyl- and alkoxy-dimethicone copolyol, polysorbate and a combination thereof;
(iii) from 30-80% w/w of a volatile solvent selected from the group consisting of hexamethyldisiloxane, isooctane and combinations thereof;
(iv) from 20% (w/w) to 40% (w/w) of water; and
(v) from 0.005% w/w to 25% w/w of a vasoconstrictor selected from the group consisting of phenylephrine, phenylephrine hydrochloride, epinephrine, epinephrine hydrochloride, tetrahydrozoline hydrochloride, an amphetamine, an antihistamine, methylphenidate, mephedrone, oxymetazoline, pseudoephedrine, psilocybin, ephedrine sulfate, and combinations thereof.

14. The composition of claim 13, comprising:
(i) from 15% w/w to 20% w/w trimethylsiloxysilicate;
(ii) from 1.5% w/w to 3.0% w/w sodium lauryl sulfate;
(iii) from 22% w/w to 30% w/w hexamethyldisiloxane and from 20-25% w/w isooctane;
(iv) from 25% w/w to 30% w/w water;
(v) from 10% w/w to 15% of a silicone acrylate; and
(vi) from 0.05% w/w to 0.25% w/w phenylephrine hydrochloride.

15. The composition of claim 14, further comprising from 3% w/w to 10% w/w of tranexamic acid.

16. The composition of claim 1, wherein the topical composition is administered to a subject in need thereof in the form of nasal swab, a single use wipe, a gel, a nasal spray, a foam, a towelette, a syringe, a dropper, a spray dispenser, a compressible bottle or tube, a spatula, a suppository insertion tube, an extrusion tube, and an inflatable member.

17. A method of treatment or prevention of any type of nose bleeding, including an Epistaxis disorder, the method comprising the step of topically applying to the mucosal surface of the affected nostril of a subject in need of such treatment a therapeutically effective amount of the composition according to claim 1.

18. A kit comprising a pharmaceutical liquid adhesive composition according to claim 1 and a container-applicator device suitable for storage and application of the composition to the nose, into the affected nostril.

19. The kit according to claim 18, wherein the container-applicator device comprises at least one of a single use wipe, a towelette, a syringe, a dropper, a spray dispenser, a compressible bottle or tube, a spatula, a suppository insertion tube, an extrusion tube, and an inflatable member.

20. A pharmaceutical liquid adhesive composition of claim 1, for use in preventing or treating any type of nose bleeding, including an Epistaxis disorder.
